(19) United States
(12) Patent Application Publication
(10) Pub. No.: US 2025/0042102 A1
(43) Pub. Date: Feb. 6, 2025

(54) Automated Apparatus

(71) Applicant: Cellular Origins Limited, Melbourn (GB)

(72) Inventors: Daniel Strange, Melbourn (GB); Paul Crisp, Melbourn (GB); Peter Crossley, Melbourn (GB); Martin Mottram, Melbourn (GB); Edwin Stone, Melbourn (GB)

(73) Assignee: Cellular Origins Limited, Melbourn (GB)

(21) Appl. No.: 18/576,812

(22) PCT Filed: Jul. 6, 2022
(86) PCT No.: PCT/GB2022/051739

§ 371 Date: Jan. 5, 2024

(30) Foreign Application Priority Data
Jul. 6, 2021 (GB) 2109779.5
Mar. 31, 2022 (GB) 2204725.2

(51) Int. Cl. B29C 65/00; A61M 39/14; B29C 65/18; B29C 65/82; B29L 23/00

(52) U.S. Cl. CPC B29C 66/857; A61M 39/14; B29C 65/18; B29C 65/8215; B29C 66/0246; B29C 66/5221; B29C 66/863; B29L 2023/007

(57) ABSTRACT

An automated apparatus for joining a first tube to another such tube whereby to form a fluidic path therethrough, comprising: means for engaging the tube and moving it into one or more positions to be manipulated; means for clamping a portion of the tube whereby to form a pinched portion of the tube such that the tube is fluidly sealed upstream of the pinched portion; means for removing an end section of the tube downstream of the pinched portion such that a new end of the tube is thereby formed within the pinched portion that has not previously contacted another such tube; and means for joining the pinched portion at the new end of the tube with a corresponding pinched portion of another such tube; wherein the apparatus is configured as an end effector for a robotic device.

### FIELD OF THE INVENTION

The present invention relates to an automated apparatus, which may be used for manipulating a flexible tube to facilitate fluid transfer between biological samples in an automated bioprocessing system used to perform automated cell therapy, for example. In a preferred embodiment, the invention relates to an automated apparatus that may be configured as an end effector for a robotic device.

### BACKGROUND

Therapeutics are increasingly using cells rather than small molecules as the starting point. The approaches to manufacturing these products are rapidly evolving to keep up with constantly emerging new therapies. In recent years, there has been an increased use of a number of new classes of cell therapies. One class is autologous cell therapies.

Autologous cell therapies are a promising class of therapy, which have significant clinical and commercial potential ranging from treating cancer to fixing genetic defects. These therapies involve taking cells from a patient, manipulating the cells over the course of days to weeks, and re-introducing the cells back into that patient's body to produce a therapeutic effect. The steps taken during autologous cell therapies are often complex; for example, a typical CAR-T process may involve a sequence of steps starting with a cryopreserved leukopak, thawing, washing to remove DMSO, enrichment of T cells, activation, transduction, expansion, concentration, formulation fill finish into an IV bag, and cryopreservation, with several other intermediate washing steps. To date, these processes have typically been performed with labour intensive manual processes in expensive class five cleanrooms or isolators.

Due to the complexity of bioprocessing, there is a desire to automate the (cell therapy) process while maintaining a closed system that removes the need to perform the steps in such a high-grade cleanroom. A closed system is one where there is no exposure of the process to the surrounding environment such that there can be no ingress of contaminants from the environment or cross contamination from other processes that are being performed simultaneously. There are systems that have tried to provide a solution to this, but involve a complex consumable element, which connects the sample to all the other necessary processing stations, for example via a tube that is fluidly connected to the consumable element, and provides pumping and valving to allow the steps to be performed in a particular sequence. However, these consumable elements are very complex to manufacture and install and are consequently relatively expensive, and potentially unreliable. Each consumable element needs to be individually tailored to the process being performed, making the system inflexible to modifications and expensive to adapt to new processes. Furthermore, typically only one consumable element can be operated/manipulated at once by these systems, which makes the bioprocessing expensive and space inefficient to scale up for use with multiple patients. Often, the system is still not capable of performing all the steps required for a complete bioprocessing method, and instead multiple isolated units may be operated in sequence, which means that additional labour and expertise is required to transfer the cells (e.g. patient samples) between the isolated units. This also introduces a further risk of cross contamination, and there is no simple way to validate that contamination has not occurred.

One way of forming sterile connections between tubes is tube welding, a process that is performed manually using semi-automated instruments. Sterile tube welders allow connections to be made between two tubes with closed ends without exposing the contents of either tube to the environment, and are the only widely accepted means of reusably creating (e.g. reconfiguring) closed connections within a single system. For a typical tube welding device, an operator first inserts and clamps two tubes within the device. Subsequently, the welder cuts the tubes, aligns the tubes to be attached together (such as by translation or rotation of the clamps), and presses the tubes together so that they are welded. Then the operator unclamps the tube, inspects the connection, and manually pinches the tube to remove the kink formed by the clamps, thereby allowing fluid to flow through the joint. Overall, the welding process can take 4 to 7 minutes of operator time.

However, existing tube welding systems have some disadvantages. These systems are typically very complex, large and heavy—common practice is for manufacturing facilities to have the tube welder placed on a trolley that can be wheeled up to the manufacturing process. Often, large portions of the tubes are discarded by the user during each tube welding operation, as the tubing must be brought to the trolley mounted tube welder. Furthermore, conventional tube welding systems are not additionally configured to cut through a single tube, and reseal securely the ends of the separated tubes afterward, which means that they cannot maintain closure of the contents when disconnecting tubes, and a separate piece of equipment (RF tube sealers) is typically used for this function.

Additionally, for an automated cell therapy process, hundreds of welds must be performed for each therapy without any of them failing. Even a 99% success rate for individual welds would only lead to a 33% success rate for one hundred welds, e.g. if performed in succession. Thus, to achieve an acceptable 99.9% success in such a process, each individual weld must have a success rate of 99.999%. One option is to try and minimize the number of tube welding operations required by the process; indeed, according to the ISO 23565 standard on design of equipment systems for cell therapy manufacturing, “the equipment should be designed and utilized in such a way that the number of in-process connections, such as tube welding, is minimized in order to reduce the risk of contaminations”. However, this approach merely circumvents the problem of unreliable tube welding, and therefore does not facilitate a flexible autonomous bioprocessing system that can perform many therapies, and preferably wherein multiple therapies can be performed simultaneously.

Thus, it would initially seem impractical to automate a manual tube welding system. In particular, it requires precise manual manipulation to insert the long flexible tubes into the welder correctly, to remove the tubes from the welder correctly, and to remove the kink in the tube after welding. Existing systems also require visual inspection by an operator after each weld is formed to confirm successful welding; with existing tube welders the tube is typically removed from the welder to be inspected, but this can lead to contamination of the surroundings or the contents of the tubes if the weld is not successful.

There has not been any progress in automating these steps in a way that would be reliable enough for use in a cell therapy process involving multiple connections and disconnections. Indeed, the problems discussed above are particularly apparent within systems where reliability and contamination issues are crucial, such as in bioprocessing systems.

Therefore, there is a need for a reliable way to form aseptic tube welds in order to allow the closed transfer of fluids and cell material, ideally which can maintain sterility/prevent contamination of the consumables and patient samples irrespective of whether the manipulation is performed within a sterile or non-sterile atmosphere in such a system.

### SUMMARY OF THE INVENTION

According to a first aspect, there is disclosed herein an automated apparatus for manipulating a flexible tube (e.g. for joining a first tube to a second tube/another such tube whereby to form a fluidic path therethrough), comprising: means for engaging a (first) tube and moving it into one or more positions to be manipulated; means for clamping a portion of the (first) tube whereby to form a pinched portion of the (first) tube such that the (first) tube is fluidly sealed upstream of the pinched portion; and means for removing an end section of the (first) tube downstream of the pinched portion such that a new end of the (first) tube is thereby formed within the pinched portion that has not previously contacted another such (or the second) tube.

Each tube may fluidly connect to a “consumable” containing, for example, cellular samples (of cell material), reagents or fluids, whereby each consumable is fluidly connected to an “upstream” end of the tube that provides a fluid conduit to a “downstream” (opposite) end of the tube, which is fluidly sealed by the pinched portion when not connected to another such (second) tube. As referred to herein, a “consumable” preferably connotes a “container” of said cellular samples, reagents or fluids, which are intended to be processed in a cell therapy process, for example, using the apparatus. As used herein, the term “engage” preferably refers to gripping the tube without the need for an operator to precisely position the tube in the apparatus.

In a preferred embodiment, the apparatus may be configured as an automated end effector, and more preferably as an end effector for a robotic device. Advantageously, by configuring the apparatus as an end effector in this way, the apparatus can move towards the tube to be engaged, engaging the tubing near the consumables to be welded and reducing the need for long flexible tube lengths. This significantly eases the challenge of automation as well as provides benefits to the overall bioprocessing system in terms of reduced dead volumes contained within the tubing. Preferably, the end effector is configured to receive electrical power from an external power supply via an electrical coupling provided therebetween. By providing an external power supply, it is possible to decrease the size and weight of the automated apparatus and the end effector.

As used herein, the term “robotic device” preferably connotes an automated machine or device programmed to perform specific mechanical functions, and which term preferably includes robots, collaborative robots (cobots), x-y or Cartesian-robots, robotic arms, and one or more actuators, possibly also comprising one or more robot end effectors, and will typically also include one or more sensors, microprocessors and power supply. The term “end effector” preferably connotes a tool, manipulator or similar device that may form part of any type of “robotic device”, for example as described above.

The apparatus may further comprise means for joining the pinched portion at the new end of the (first) tube with a corresponding pinched portion (e.g. at a corresponding new end) of the, or a, second tube/another such tube. The two tubes (i.e. their respective ends) are joined together preferably to form a butt weld between them. The means for joining may be configured to press the pinched portion of the first tube into the pinched portion of the second tube, thereby forming the butt weld.

Preferably the means for removing an end section of the tube comprises a heated cutting element configured to cut through said pinched portion of the (or each) tube, thereby melting the pinched portion at the new end of said tube such that a butt weld can be formed between the ends of the tubes when joined together while still melted (i.e. before the material of the tubes cools and solidifies). The cutting element may comprise a blade, ribbon, or wire that is arranged to be heated. Preferably, the cutting element is a blade comprising a copper wafer. The heated cutting element may be configured to cut and/or melt the tube for a predetermined time period and/or may have a predetermined heat profile. Where the heated cutting element is used to cut through a tube (without subsequent reconnection to another such tube), the heated cutting element may be configured to cut and/or melt the tube for a different predetermined time period, and/or may use a different predetermined heat profile. In other words, the heat profile for connection of two tubes may be different to the heat profile for disconnection of two tubes.

Preferably, the means for joining the pinched portion is configured to translate the (or each) tube in a direction coaxial with a longitudinal length of the (or each) tube. In this way, the apparatus may engage the tube at one position along its length and clamp the tube at a different position along its length; this allows the length of the end section of the tube to be minimized prior to removal, thereby reducing the amount of wasted tubing. Furthermore, it may be preferable to engage the tube at a fixed or predetermined location in space (such as where the tube connects to a corresponding container), and then translate the tube until the means for joining reaches the end portion of the tube; this may increase reliability of the apparatus in engaging the tube, since the apparatus no longer needs to engage the tube at an arbitrary position in space.

Preferably, the means for clamping a portion of the (first) tube is arranged to form a pinched portion of the (first) tube towards an end of the (first) tube. In this way, the amount of tubing that is removed may be reduced, thereby decreasing the amount of wasted tubing.

Preferably the means for engaging the (first) tube is operable to retain the end section of the (first) tube that is removed downstream of the pinched portion. Advantageously, this may prevent discarded lengths of downstream tubing from accumulating around the apparatus, which may interfere with its operation. By retaining the end section that is removed, the apparatus may be configured to dispose of the end section at a particular predetermined location.

Preferably, said means for engaging the (first) tube is configured to align it with another such (or second) tube such that the new ends of each tube, formed by removal of the end section of each tube, respectively, share a common plane, for example such that the first and second tubes are adjacently arranged. Preferably, the first and second tubes are arranged such that one of the first or second tubes is inverted, for example such that the “downstream” end of the first tube extends towards the “upstream” end of the second tube, and vice versa.

The means for clamping the tube may be further configured to seal the tube. For example, it may be advantageous to provide sealing of the tube during a disconnection process (in addition to sealing provided by the heated cutting element). It may be advantageous to seal the tube (preferably the downstream “end” section) during a connection process, such as before the end section is discarded. Additionally, if a join is not successful, the tube may be fully sealed before being unclamped and re-welded, thereby reducing the risk of contamination. By sealing the tube with the clamping means, the tube may be sealed across a wider area, thereby providing a more robust seal of the tube. The means for clamping may comprise a heat source to melt the tube once clamped, thereby heat sealing the clamped tube. The heat source may be a resistive element or may be an electromagnetic source such as an RF source.

The apparatus may further comprise a sensor configured to determine whether the first tube and/or the second tube are engaged and/or aligned in the means for engaging the tube. The sensor may include a camera for visually inspecting the engagement and/or alignment of the tubes. The sensor may include a mechanical sensor, a pressure sensor, a capacitive sensor, or any suitable sensor or combination of sensors that may determine the presence and/or position of a tube in the means for engaging.

Preferably, the apparatus further comprises means for manipulating the tube, once joined with said another such tube, to release the pinched portion whereby to establish a fluidic path between the joined tubes. In this way, fluid is able to pass through the joined tube to enable a controlled transfer of fluid between corresponding containers.

The apparatus may maintain engagement with the tube during operation of one or more of: the means for engaging the tube, the means for clamping, the means for removing, the means for joining, and/or the means for manipulating the tube to release the pinched portion. Advantageously, the first tube may be joined to the second tube without the apparatus releasing and re-engaging either or both of the tubes. This reduces the risk of misalignment of the tubes during any part of the joining operation. For example, by maintaining engagement throughout the process, the pinched portion of the tube may not need to be realigned with the means for manipulating the tube to release the pinched portion.

More preferably, the means for manipulating the tube comprises an automated pinch-release mechanism arranged to press on an edge of the pinched portion of the tube thereby to release the pinched portion and open up the welded tubes whereby to form a fluidic path therethrough.

According to a second aspect, there is disclosed herein an apparatus for releasing a pinched portion of tube, comprising an automated pinch-release mechanism arranged to press on an edge of the pinched portion of the tube thereby to release the pinched portion and open up the welded tubes whereby to form a fluidic path therethrough.

As used herein the term “automated pinch-release mechanism” preferably connotes a mechanism that may function without any operator intervention; more specifically, an operator is not required to (directly or indirectly) press against the pinched portion, and preferably does not need to initiate or trigger the pinch-release mechanism to release a particular pinched portion. As used herein, the “edge of the pinched portion” refers to a border (or “fold”) between the opposing two sides of the tube once it has been pinched or flattened by the means for clamping. By automating the pinch-release mechanism, a full tube welding operation may be completed completely autonomously. The automated pinch-release mechanism may be located on an end effector of a robotic device, which may be the same end effector and robotic device described above or may be a separate end effector and/or a separate robotic device.

The following optional features may apply to the apparatus of either the first or second aspect (or both).

Preferably, the pinch-release mechanism is arranged to compress the pinched portion of the tube against a seat portion of one or more of the means for engaging and moving the tube and the means for clamping a portion of the tube. In this way the seat portion provides an opposing force to push against the tube during use of the pinch-release mechanism. Additionally, the seat portion may maintain alignment of the tube during unpinching of the tube and/or during other operations of the apparatus.

Preferably, the pinch-release mechanism comprises a first release element and a second release element, the first and second release elements arranged to engage with opposing edges of the pinched portion of the tube at an interface between the new ends of the first and second tubes when joined together. The first and second release elements may grip (engage) the tube when the tube is fully released by the means for engaging the tube.

The pinch-release mechanism may be arranged to press on an edge of the pinched portion by moving along a straight path. In this way, the pinch release mechanism may release the pinched portion without applying a shearing or twisting force to the tube that may damage the weld. Preferably, the straight path is perpendicular to the direction in which the tube is clamped by the means for clamping the tube.

Preferably, the apparatus further comprises means for stretching the interface between the new ends of the first and second tubes when joined together (e.g. applying a tensile force to the respective tubes to gently pull them apart) to verify the integrity of the join, preferably wherein said means for joining is further configured to provide said means for stretching, thereby to perform quality control or quality assurance (QC) checks.

Preferably, the apparatus further comprises means (e.g. a machine vision system) for inspecting the interface between the new ends of the first tube and another such (or second) tube when joined together, for example to verify the integrity of the join thereby to perform quality control or quality assurance (QC) checks, wherein said join is preferably a butt weld. The means for clamping may be configured to keep the tube clamped shut on at least one side, and preferably both sides of the join during the inspection, thereby maintaining a closed system until integrity of the join is verified. The apparatus may be configured to retain the join at a fixed location relative to the means for clamping during inspection of the join; in this way, there is a reduced risk of damaging a join prior to inspection, and a reduced risk of fluid moving between the upstream and downstream sections of the tubes. The means for inspecting the interface may also provide the sensor configured to determine whether the first tube and/or the second tube are engaged and/or aligned in the means for engaging the tube. Alternatively, separate sensors may be provided for inspecting the interface and for verifying engagement and alignment.

Preferably, the means for engaging a tube is provided by a tube gripping mechanism comprising a pair of jaws operable for relative movement between an open configuration for receiving the tube therebetween and a closed configuration for the tube therebetween. The means for clamping a portion of the tube may also be provided by the tube gripping mechanism, where the closed configuration of the pair of jaws is configured to clamp the tube therebetween. Where the closed configuration of the jaws is used to clamp the tube, a position between the open configuration and the closed configuration may be used to engage the tube. Alternatively, the means for clamping may be provided in other ways and/or may be provided by a different gripping mechanism to the means for engaging the tube.

More preferably, the tube gripping mechanism may comprise a plurality of pairs of gripping elements arranged adjacently, each pair of gripping elements comprising a first gripping element and a second gripping element, and each pair of gripping elements being configured for movement between said open and closed configurations.

According to a third aspect, there is disclosed herein a tube gripping mechanism comprising a plurality of pairs of gripping elements arranged adjacently, each pair of gripping elements comprising a first gripping element and a second gripping element that are operable for relative movement between an open configuration for receiving a tube therebetween and a closed configuration for engaging and clamping a portion of the tube therebetween, wherein each pair of gripping elements is configured for movement between said open and closed configurations.

As used herein, the term “grip” or “gripping” may connote both lightly gripping (e.g. for engagement or manipulation of the tube) and tightly gripping (e.g. for clamping or pinching the tube shut for a welding operation), depending on the context. It will be appreciated that each pair of gripping elements may be operated at an intermediate position between said open and closed configurations.

The following optional features may apply to the apparatus of either the first or second aspect (or both) or the mechanism of the third aspect.

Preferably, the tube gripping mechanism comprises a seat portion to abut against the tube when it is located between the opposing pairs of gripping elements. In this way, the seat portion may maintain alignment of the tube when located in the gripping mechanism. This means that the tube is always engaged and clamped in the same way between the opposing pairs of gripping elements, thereby improving consistency and reliability of the joining between the tubes.

Preferably, both of the opposing pairs of gripping elements are configured to move, so that the tube may remain centred throughout movement between the open and closed configurations, which may ensure consistent alignment of the tube when gripped and/or clamped.

Preferably, at least one of the gripping elements is moved by a corresponding cam. Using cams to actuate the gripping elements may enable more reliable and precise positioning of the gripping elements.

Preferably, a first pair of gripping elements and an adjacent second pair of gripping elements are spaced apart to provide a gap for receiving therebetween said means for removing an end section of the tube. In this way, the pairs of gripping elements do not need to be adjusted in order to make space for the means for removing an end section (e.g. a cutting blade).

Preferably, a first pair of gripping elements is configured to engage a first portion of the end section of tube before a second pair of gripping elements is configured to engage an adjacent second portion of the end section of the tube, whereby to urge fluid in the tube away from the first portion of the tube prior towards the second portion of the tube prior to operation of (said) means for removing an end section of the tube arranged to remove a portion of the end section of the tube on the other side of the first pair of gripping elements. Additionally, or alternatively, a first pair of gripping elements that is closer to said gap is configured to clamp the tube before a second pair of gripping elements that is further from said gap, thereby removing fluid from the clamped portion of the tube prior to operation of the means for removing the end section of the tube. By removing (or “pumping”) fluid from the clamped portion of the tube in this way, fluid loss is reduced, and the fluid is less likely to interfere with the welding process. This increases the reliability of the apparatus or mechanism when creating wet-wet welds (i.e. welds between tubes still containing some fluid).

Preferably, the gripping mechanism is controllable to clamp different parts of said portion of tube while releasing other parts of said portion of tube. For example, adjacent opposing pairs of gripping elements may be in different intermediate positions between said open and closed configurations so that some parts are gripped (engaged or clamped), and other parts are released. Furthermore, adjacent opposing pairs of gripping elements may open and close at different times (e.g. in a pre-determined sequence). In other words, the plurality of opposing pairs of gripping elements may move out of phase with each other. This may be achieved by actuating each opposing pair of gripping elements with a separate cam, where each cam is rotationally offset to an adjacent cam.

Advantageously, by providing a gripping mechanism that can selectively grip parts of the portion of the tube while releasing other parts, the gripping mechanism may gradually engage the tube and straighten it out, rather than bunch it up. Preferably, the gripping mechanism is controllable to retain the portion of tube between at least one pair of gripping elements in the closed configuration while at least one different pair of gripping elements are moved to the open configuration to release part of said portion of tube such that said part of said portion of tube is exposed. The apparatus or mechanism may further comprise means for inspecting said released or exposed part of said portion of tube. In this way, it may be possible to determine whether the weld is successful while keeping the upstream portion of the tube isolated from the join, thereby preventing exposure of the contents of the tube to the surroundings. This also allows inspection of the join without removing the tube from the apparatus or mechanism, which allows the tube to be re-clamped and re-welded immediately without needing to re-engage the tube.

Preferably, the, or a, means for manipulating the tube to release the pinched portion is arranged to compress (e.g. apply a compressive force to) said released or exposed part of said portion of tube. In this way, it may be possible to release the pinched portion without completely releasing the tube from the apparatus or mechanism and without needing to re-engage the tube. This may substantially improve reliability, as in this way the pinching mechanism can be configured to always act on the same portion of the weld, and there is substantially reduced risk of the tubing twisting or otherwise moving prior to the pinch-release.

Preferably, the plurality of pairs of gripping elements of the gripping mechanism are controllable to move an engaged tube in a direction substantially perpendicular to a longitudinal length of the tube relative to the gripping mechanism. In this way, the distance between a first tube and a second tube (e.g. held in a second gripping mechanism) may be changed before and/or after removing the corresponding end sections; this may allow a smaller blade to be used for simultaneously cutting both tubes. This may also prevent the tubes becoming accidentally welded due to proximity to each other; preferably the first tube remains at least 4 mm from the second tube during welding. Additionally, this may reduce the subsequent time taken to align and join the tubes after the end sections are removed, which may improve the quality of the weld (such as ensuring the tubes are hotter when connected together).

Preferably, said means for manipulating the tube to release the pinched portion is arranged to engage the tube between a gap provided between adjacent gripping elements, for example the gap for receiving therebetween said means for removing an end section of the tube. The means for removing an end section of the tube may also provide the means for manipulating the tube to release the pinched portion; for example, where a heated blade is used to remove the end section of the tube, the blade may be allowed to cool before being through the gap to release the pinched portion.

According to a fourth aspect, there is disclosed herein an automated and/or robotic system comprising any apparatus or mechanism described herein. The robotic system may comprise one or more robotic devices.

Any of the apparatus or mechanism described herein may be configured as an automated end effector, for example a robotic end effector for a robotic device.

As used herein, the term “automated system” preferably connotes a system operated and/or controlled by automation, and which term preferably includes one more of the following: robotic devices, conveyers, one or more actuators configured to engage and/or move containers or indeed any combination of these features that are capable of moving and/or manipulating the containers and/or tubes within the system.

As used herein, the term “bioprocessing” preferably includes cell therapy, such as autologous and allogenic cell therapies, as well as vaccines and (small batch) bioprocess, for example.

As used herein, the term “aseptic connection” preferably connotes a connection where contents of the respective containers being connected are not exposed to the surrounding air or atmosphere. The term “aseptic connection” may equivalently be referred to as a “closed connection” or a “sterile connection”, for example.

As used herein, the term “fluid” preferably connotes liquid and/or gas, and may further include material such as cell material contained therein

It will be understood by a skilled person that any apparatus feature described herein may be provided as a method feature, and vice versa. It will also be understood that particular combinations of the various features described and defined in any aspects described herein can be implemented and/or supplied and/or used independently.

Moreover, it will be understood that the present invention is described herein purely by way of example, and modifications of detail can be made within the scope of the invention. Furthermore, as used herein, any “means plus function” features may be expressed alternatively in terms of their corresponding structure.

### BRIEF DESCRIPTION OF THE DRAWINGS

One or more embodiments will now be described, purely by way of example, with reference to the accompanying figures, in which:

FIG. 1 shows a schematic diagram of an automated (bioprocessing) system;

FIG. 2a shows an embodiment of an apparatus comprising a robotic arm with an end effector according to the present invention, and FIG. 2b shows a cut-away (internal) view of the end effector of FIG. 2a;

FIGS. 3a to 3m show cut-away views of the end effector of FIG. 2 at various stages of manipulating a flexible tube;

FIG. 4a shows a cut-away rear view of the end effector of FIG. 3, and FIGS. 4b and 4c show a set of cams arranged to control a plurality of jaws of the end effector;

FIGS. 5a to 5f are schematic diagrams showing gripping elements that form the jaws of the end effector of FIGS. 2 and 3 at various stages of disconnecting a flexible tube; and

FIGS. 6a to 6c are schematic diagrams showing gripping elements of the end effector moving a tube longitudinally through the end effector.

### DETAILED DESCRIPTION

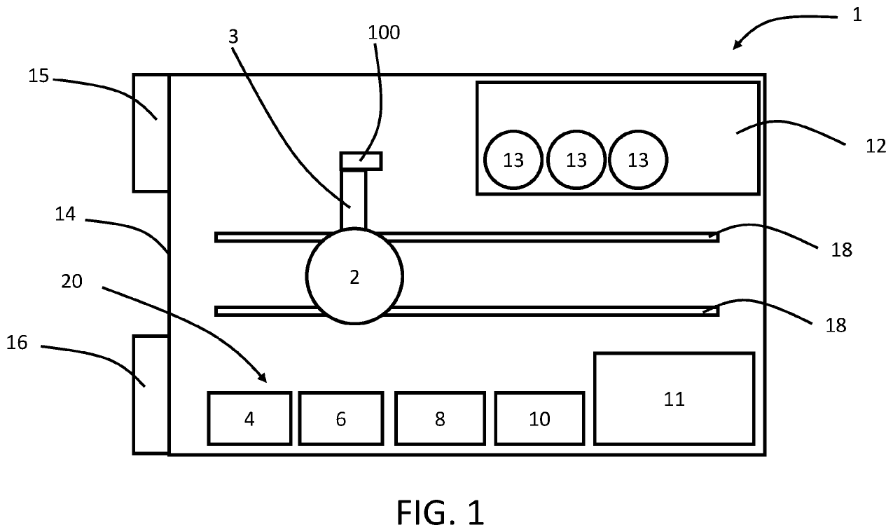

An exemplary bioprocessing system 1 is shown in FIG. 1. The system 1 has a series (e.g. a “plurality”) of processing stations 20 configured to perform processing steps for bioprocessing, and an (automated) system for automating (at least part of) the process.

In this exemplary system 1, the processing stations 20 include a thawing station 4, a centrifuge 6, a magnetic cell separator 8, a controller rate freezer 10, and a refrigerator 11, though additional and alternative stations 20 (not shown) for processing can be installed depending on the specific process being performed by the system 1.

The processing stations 20 may include any combination of a concentration station, a cryopreservation unit, a washing station, a cell enrichment station, a cell expansion station, a cell selection station, stations for determining cell count, cell viability or cell type, or stations for any other suitable processing or analysis step. The system 1 also has an incubator 12 that is large enough to contain and incubate multiple consumables 13 at a time, including under perfusion.

For example, the incubator 12 may be capable of storing twenty consumables 13 and operate at around 37° C., though the number of consumables 13 can be chosen to meet the needs of the particular bioprocessing to be performed. Each consumable 13 may contain cellular samples, reagents or fluids, and each consumable 13 connects to a first “upstream” end of a tube (150 not shown) which leads to a second “downstream” end of the tube 150, which is fluidly sealed when unconnected (or “free”). Thus, as referred to herein, a “consumable” may be in the form of a “container”, which may for example hold cell material to be processed in a cell therapy process.

All of the consumables 13 and reagents may be pre-loaded in the system 1 before a particular bioprocess begins, though additional reagents can be added throughout the process if required (for example at day 7 of a 10-day therapy process). The additional reagents may be required for reactivation of cells, or to add additional media to the consumables 13 for example.

A particular bioprocess may be defined by a bioprocessing workflow, and preferably the system 1 can be configured to carry out several bioprocessing workflows. For example, the system 1 can carry out the same bioprocessing workflow in parallel for multiple patient samples, or it can carry out different bioprocessing workflows in parallel for multiple patient samples. Each bioprocessing workflow may use a different subset of the processing stations 20 in the system 1. In a preferred embodiment, the system 1 comprises stations 20 to perform concentrations, washing and incubation processes.

The system 1 comprises an automated system configured to install one or more consumables 13 into each of the series of processing stations 20 and to move the consumables 13 between stations 20. The automated system includes a robotic device 2 that can move the consumables 13 between the various stations 20, and can manipulate the tubes 150 (e.g. fluid conduits) connecting to each of the consumables 13.

The robotic device 2 may be mounted on rails 18, which allows the robotic device 2 to have access to all areas of the system 1 such as the stations 20. The robotic device 2 may also be mounted on an autonomous mobile robot such that it can autonomously move around a factory floor to perform tasks such as sampling from bioreactors. The robotic device 2 may be configured as a co-operative robot (“cobot”). The robotic device 3 may be an XY or Cartesian robot, or a robot on a gantry, for example. The robotic device 2 may have a robotic arm 3 for manipulating the consumables 13 and tubes 150, as shown here, or may include a conveyer belt, one or more actuators, or any combination of the above aspects.

The automated system is configured to manipulate a fluid connection between a first consumable 13 and a separable second consumable whereby to create an aseptic connection that enables a controlled transfer of fluid or cell material between the first consumable 13 and the second consumable 13. Here, the robotic device 2 is used to form (or manipulate) fluid connections between the tubes 150 so that separate consumables 13 can be connected together.

The connection between two tubes 150 is preferably performed by an apparatus such as a tube welder, which may be located at one of the processing stations 20. The apparatus may be configured as an end effector 100 located on the robotic arm 3 and/or on a XY or Cartesian robot or a robot in a gantry, for example. The apparatus (e.g. end effector 100) may equivalently be referred to as a “manipulator” or “tool”, or more generally as a “tube connecting means”. It will be appreciated that any features of the tube connecting means (e.g. end effector 100) described herein may be implemented as a static tube welding apparatus, which may form part of the bioprocessing system 1 or may be provided as a separate piece of apparatus. The connections between separate tubes 150 are made aseptically such that the contents of the consumables 13 and tubes 150 are never open or exposed to the surrounding air or atmosphere at any stage.

The fluid connections are also reconfigurable, such that two separate tubes 150, once joined together, can later be disconnected and reconnected to different consumables 13 as many times as necessary in order to perform the required bioprocessing method. In other words, the automated system is configured to create an aseptic connection that can be disconnected after the transfer of fluid or cell material is complete to enable a further such fluid connection to be manipulated between the first consumable and a separable third consumable. Preferably, subsequent connections are made at a different position along the tube 150 to previous connections. As mentioned above, during both the connection and disconnection, the consumables 13 and their respective tubes 150 never have their contents exposed to the surrounding air or atmosphere such that a controlled transfer of fluid and/or cell material occurs only between the consumables 13 that are connected together via their respective tubes 150. As a result, it is not strictly required to have a sterile atmosphere around stations 20, consumables 13, or robotic device 2, but an enclosure 14 may be provided to prevent access by operators and/or to provide a sterile atmosphere or otherwise control the environment for example by controlling the temperature, light levels or other conditions.

The system 1 preferably also has a pumping unit (not shown) arranged to pump fluid along the tubes 150 once the robotic device 2 has successfully connected two consumables 13 via their respective tubes 150. The pumping unit may be located on the robotic arm 3 or may be a static component placed at one of the stations 20 into which the tubes 150 are placed by the robotic arm 3 for pumping to occur. While a pumping unit is preferred, transfer of fluids and cell material could for example be effected by way of gravity, or by addition of gas via a sterilising filter.

It is no simple matter to automate a tube welding process, as will be understood from the following challenges that must be addressed. Indeed, there are several reasons that would initially make tube welding appear incompatible with automation.

Firstly, tube welding requires the handling of long flexible tubes, which are extremely challenging for an automated apparatus to handle. In particular, flexible tubing does not always stay within a well-defined deterministic location once moved there, e.g. by a robotic device, making engagement, alignment and orientation of the tubes challenging. In particular, to engage flexible tubing in free space and self-load the tubing into a tube welder without human intervention is very difficult. When the end of a sufficiently long flexible tube, constrained at one end, is manipulated at the other, the path defined by the tube will have a number of non-unique solutions as to what the path between the constrained end and the manipulated end will take-up, dependent on the internal stress in the tube. Such a system has many degrees of freedom. Such applications are ideal for humans but very challenging for automation and therefore typically left for human operators. Furthermore, since weld strength is very dependent on precise positioning and compression of the tube, any errors in engagement of the tubing by a robot may lead to failure of the weld. Thus, it would not have been feasible simply to take an existing tube welding apparatus and automate it on a robotic end effector to engage with tubing.

Secondly, the size and weight of typical tube welders make them very inappropriate for incorporation into an end effector for a robotic arm. Typical tube welders weigh at least 2.5 kg and usually weigh more than 5 kg. Since robotic arms appropriate for use in bioprocessing systems are not generally designed to be capable of handling heavy payloads (e.g. more than 5 kg), any weight allowance used to carry the weight of the end effector may limit the effectiveness of the robotic device when manipulating tubes and other objects in the bioprocessing system. Furthermore, typical tube welders are usually bulky with lengths in excess of 30 cm; when incorporated into an automated bioprocessing system, the end effector needs to be relatively small in order to engage with tubing and other instruments in the bioprocessing system.

Thirdly, a tube welding operation requires a large number of steps, which must all be completed reliably and must be fully automated without any manual intervention. This means that the end effector must include features for engaging tubing (as discussed above), detecting the presence of tubing, clamping the tubing in an area free of fluid, welding the tubes, unpinching the tube, and performing quality control (QC) of the weld. As previously noted, to insert the tubes into the welder correctly, to remove the tubes from the welder correctly, and to remove the kink in the tube after welding requires precise manual manipulation.

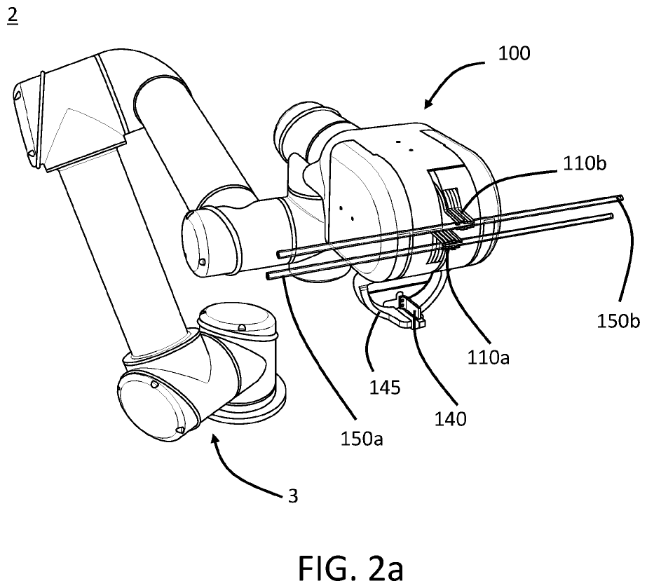

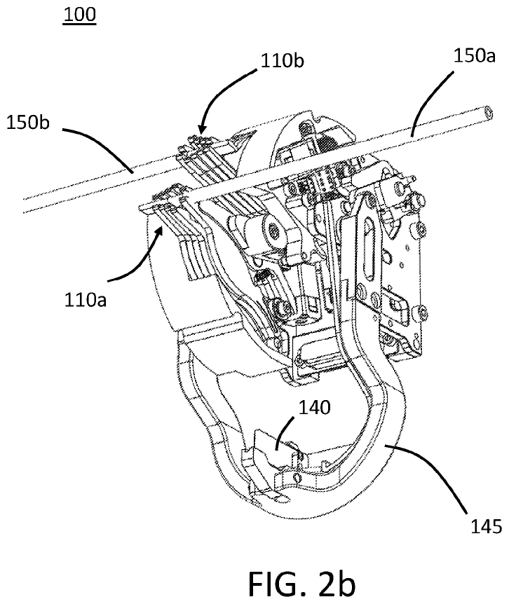

With reference to the robotic device 2 shown in FIG. 2a, a preferred embodiment of an automated apparatus 100 for joining a first tube 150a to another such tube 150b whereby to form a fluidic path therethrough will now be described. In this exemplary embodiment, the robotic device 2 comprises a robotic arm 3 with an end effector 100 configured to provide said apparatus 100. Additionally, or alternatively, the apparatus 100 may be described as being suitable for manipulating fluidic aseptic connections between two tubes 150, whereby to form a fluidic path therethrough.

The internal components of the end effector 100 are shown in the cut-away view of FIG. 2b. The end effector 100 has two gripping mechanisms 110a, 110b each for gripping a respective tube 150a, 150b. The end effector 100 is configured to fluidly connect a first tube 150a and a second tube 150b together while maintaining a seal between the inside of the tubes 150a, 150b and the surroundings (i.e. the contents of the tubes and consumables 13 are not exposed to the atmosphere). Each tube 150a, 150b may fluidly connect to a respective consumable 13 (not shown).

As used herein, the term “upstream” refers to a direction along the tubes 150a, 150b leading towards a respective consumable 13. Similarly, the term “downstream” refers to a direction along the tubes 150a, 150b leading towards the “free” end of the tubes 150a, 150b. The first and second gripping mechanisms 110a, 110b may also be used to perform other steps in a cell therapy process, such as to move consumables 13 between processing stations 20. As will be described later in more detail, the end effector 100 also comprises a blade 140 attached to a blade arm 145 that can be moved along a cutting plane 141 (not shown) thereby cutting through the tubes 150a, 150b. When cutting occurs, the tubes 150a, 150b are preferably orientated parallel to each other and perpendicular to the cutting plane 141. The direction parallel to the tubes 150a, 150b may be referred to as a longitudinal direction.

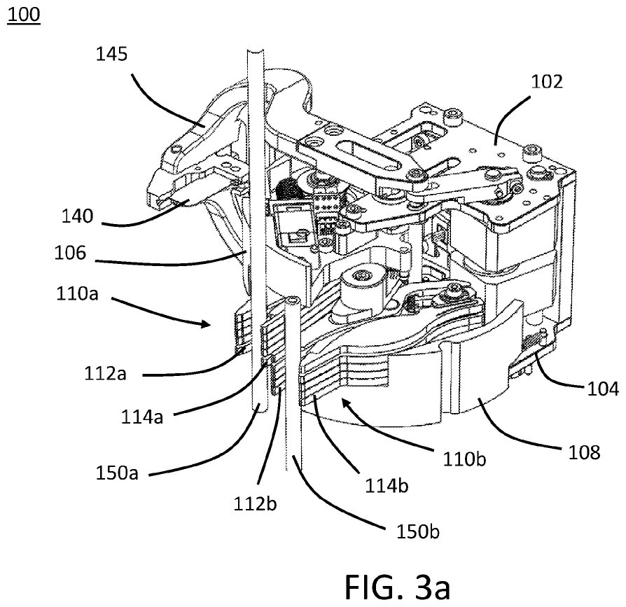

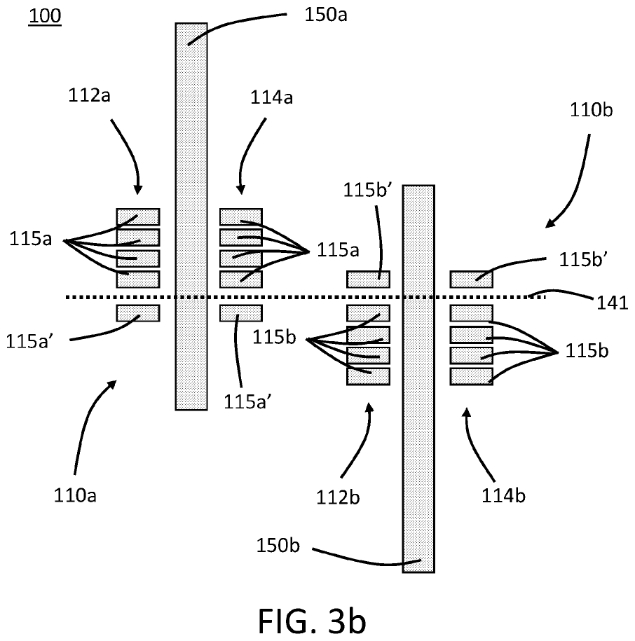

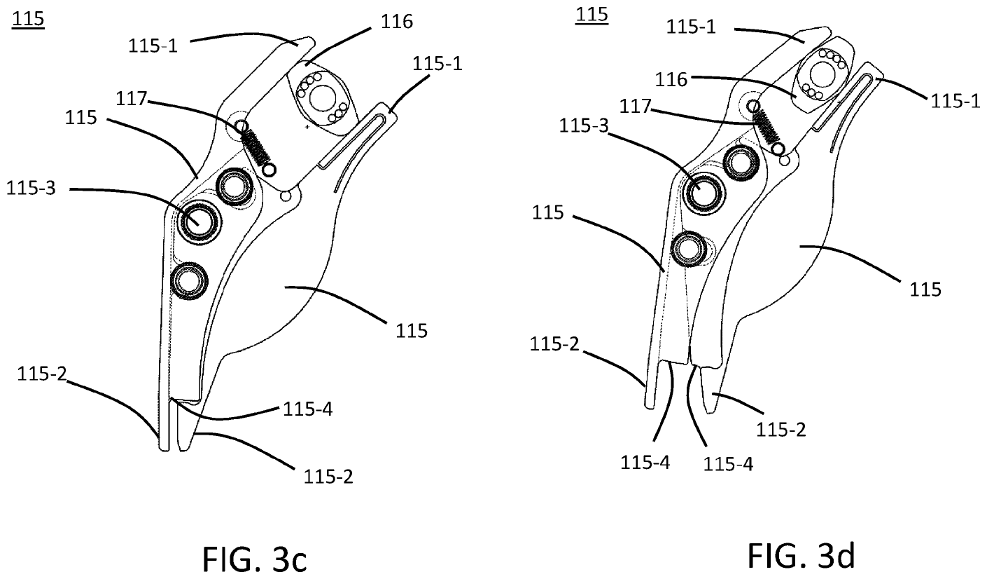

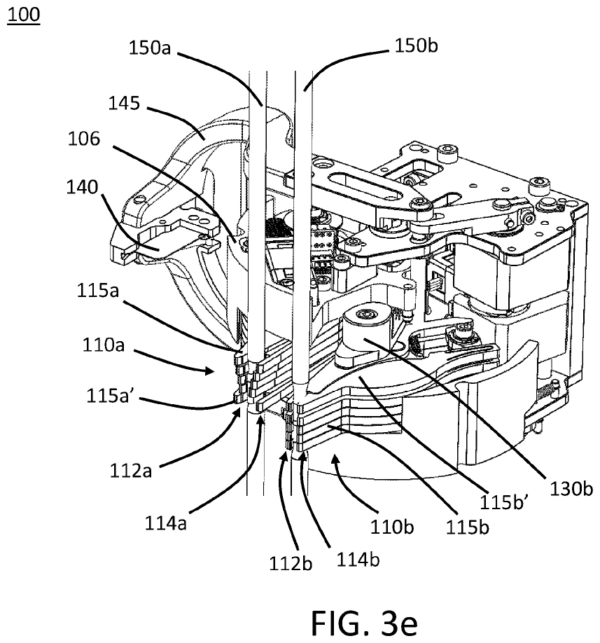

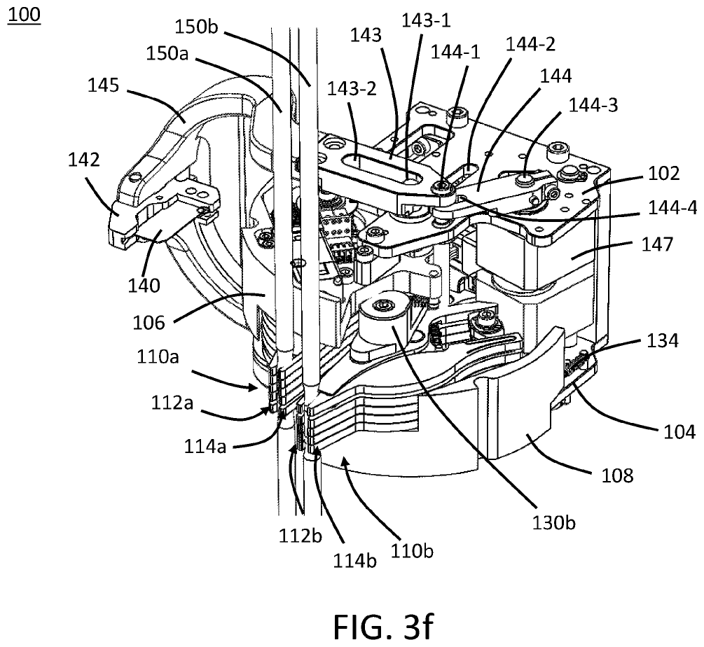

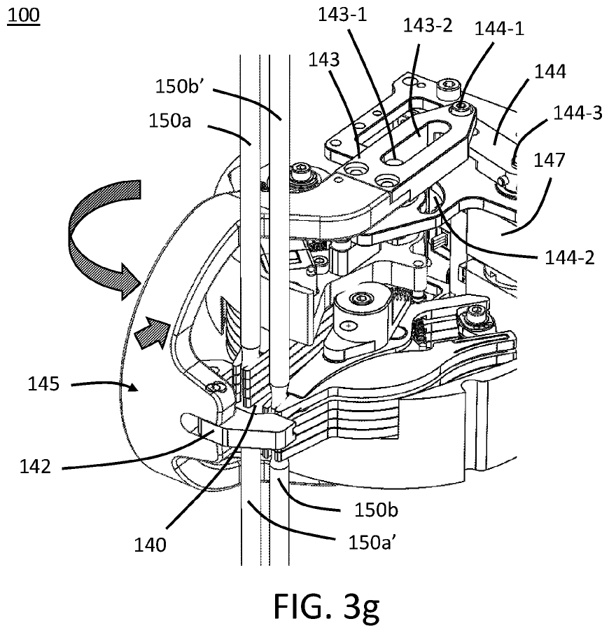

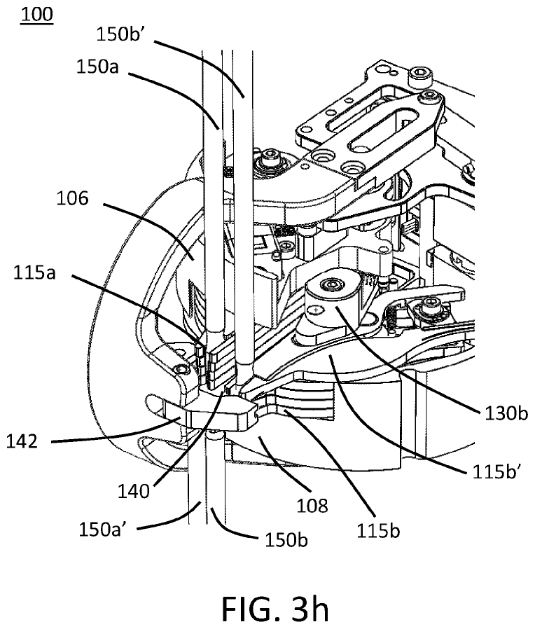

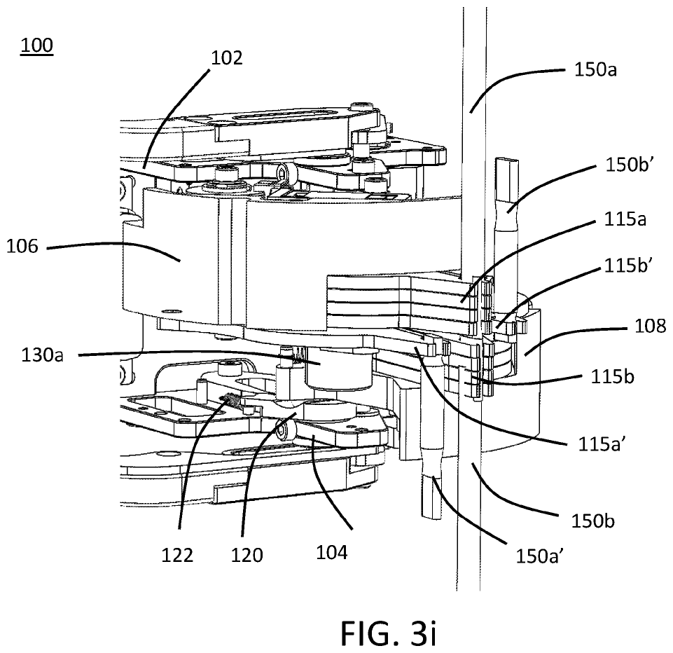

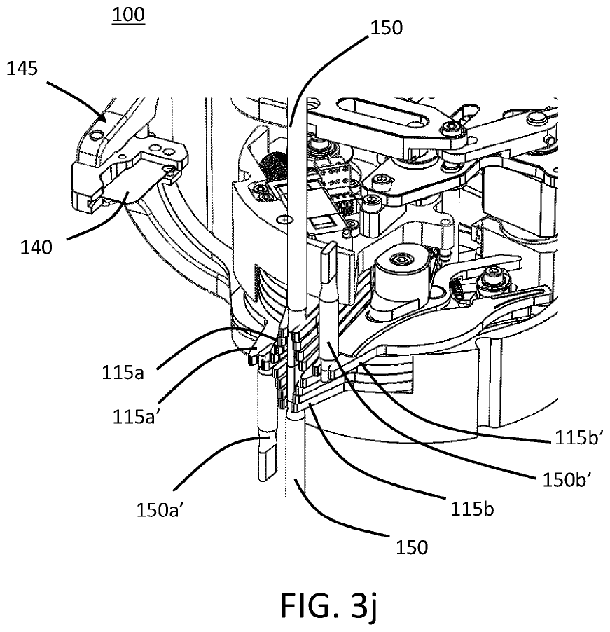

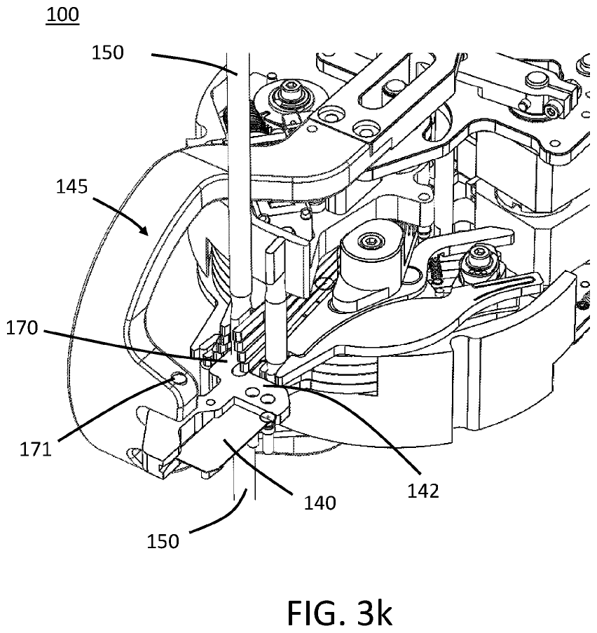

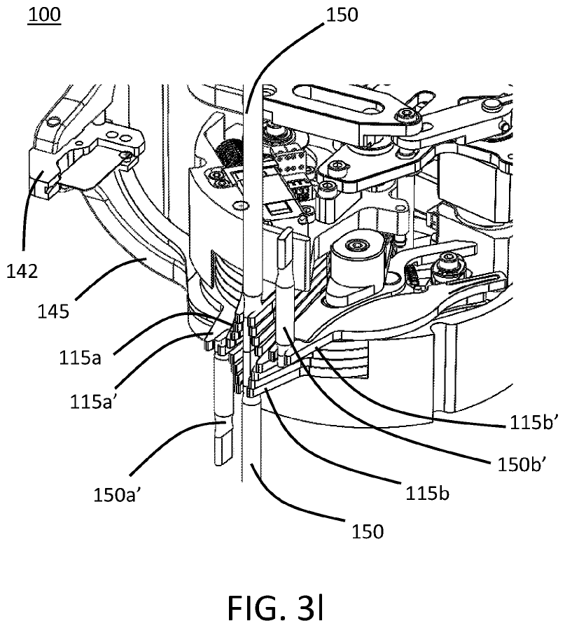

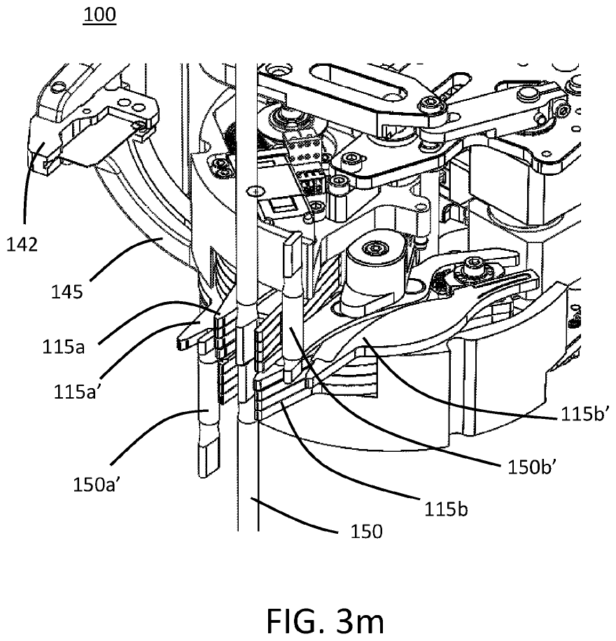

With reference to FIGS. 3a to 3m, the operation of the end effector 100 will now be described in detail. As shown in FIGS. 3a and 3b, the first gripping mechanism 110a has a first jaw 112a and a second jaw 114a. Similarly, the second gripping mechanism 110b has a first jaw 112b and a second jaw 114b. The jaws 112, 114 are operable to grip the respective tube 150a, 150b therebetween. The term “clamp” or “engage” may also be used to refer to the gripping of the tubes 150a, 150b between the jaws 112, 114.

When the gripping mechanisms 110a, 110b are operated to join (e.g. butt weld) two tubes 150a, 150b together, the tubes 150a, 150b may be tightly gripped or “clamped” tightly between the jaws 112a, 114a; 112b, 114b formed by each of the gripping mechanisms 110a, 110b so as to pinch them fully shut. On the other hand, when the tubes 150a, 150b are being manipulated inside the bioprocessing system 1, they may instead be lightly gripped or “engaged” between the jaws 112a, 114a; 112b, 114b of the gripping mechanisms 110a, 110b such that fluid may still pass through the gripped region of the tubes 150a, 150b.

The jaws 112, 114 of each gripping mechanism 110 may each comprise a plurality of gripping elements 115 (also referred to herein as “finger elements” or “fingers”), which may be individually actuated to grip the tubes 150. An opposing pair of gripping elements 115 may therefore be referred to as forming a pair of said jaws 112, 114. A pair of jaws 112, 114 may further comprise a plurality of opposing pairs of gripping elements 115. The gripping elements 115 on the jaws 112, 114 may be arranged adjacent one other, thereby providing an “array” of adjacent gripping elements 115 forming a pair of jaws 112, 114. As depicted in FIGS. 3a to 3m, each pair of jaws 112, 114 comprises five opposing pairs of gripping elements 115. However, any number of opposing pairs of gripping elements 115 may be used, though preferably each gripping mechanism 110 comprises at least one pair of opposing gripping elements 115 positioned either side of the cutting plane 141. If only one pair of opposing gripping elements 115 is provided on each side of the cutting plane 141, for example, a separate gripping unit (not shown) may be used to engage and/or manipulate the tubes 150a, 150b to locate them between a pair of jaws 112, 114 on the apparatus 100.

Each opposing pair of gripping elements 115 is movable between a closed configuration in which the gripping elements 115a, 115b pinch or “clamp” the tubes 150a, 150b and an open configuration, where the tubes 150a, 150b are received or released by the gripping elements 115a, 115b. Each opposing pair of gripping elements 115 may be moved to any position between the open and closed configuration, such as to engage the tube 150 without pinching it closed.

In FIG. 3a, the tubes 150a, 150b are each disposed between a pair of jaws 112, 114 of a respective gripping mechanism 110 but the jaws 112, 114 are not yet actuated to grip the tubes 150a, 150b. In other words, all of the opposing pairs of gripping elements 115a, 115b are in the “open configuration”. The end effector 100 comprises a first baseplate 102 and a second baseplate 104. The end effector 100 may also comprise a first mounting plate 106 and a second mounting plate 108, which may move relative to the baseplates 102, 104. The first gripping mechanism 110a is mounted to the first mounting plate 106, and the second gripping mechanism 110b is mounted to the second mounting plate 108. As will be described later, motors or other actuators may also be mounted to the baseplates 102, 104 or the mounting plates 106, 108 so as to actuate parts of the end effector 100. Alternatively, the actuators and other parts of the end effector 100 may be mounted elsewhere without affecting its overall operation. The orientation of the end effector 100 may be altered by the robotic arm 3. For example, FIGS. 2a and 2b show the end effector 100 holding the tubes 150a, 150b horizontally, whereas FIGS. 3a to 3l show the end effector 100 holding the tubes 150a, 150b vertically.

FIG. 3b depicts a cross section through the end effector 100. The cutting plane 141 where the tubes 150 will be cut by the blade 140 is shown using a dotted line. Both the first and second gripping mechanisms 110a, 110b have a pair of opposing gripping elements 115 on both sides of the cutting plane 141. In other words, both gripping mechanisms 110a, 110b have at least gripping element 115a, 115b from each of the first jaw 112a, 112b and second jaw 114a, 114b on both sides of the cutting plane 141. The space between adjacent gripping elements 115 may be larger for the gripping elements 115, 115′ located either side of the cutting plane 141 than those gripping elements 115 located on the same side of the cutting plane 141 in order to allow a gap for the blade 140 to pass therebetween. As will be described later in more detail with reference to FIG. 3i, a lifting mechanism 120 may be used to increase or decrease this gap.

In the present embodiment, there is one gripping element 115a′, 115b′ on each pair of jaws 112, 114 that is positioned downstream of the cutting plane 141 for their respective tube 150a, 150b; these gripping elements 115a′, 115b′ may be referred to hereinafter as the “downstream” gripping elements 115a′, 115b′. The present embodiment has four opposing pairs of gripping elements 115a, 115b on each of the jaws 112, 114 that are positioned “upstream” of the cutting plane 141; these gripping elements 115a, 115b may be referred to hereinafter as the upstream gripping elements 115a, 115b. There may be a different number of upstream gripping elements 115a, 115b and/or downstream gripping elements 115a′, 115b′ forming the pair of jaws 112, 114 on either or both of the gripping mechanisms 110a, 110b.

Referring now to the “gripping elements” hereinafter as “fingers”, the region of the fingers 115 which makes contact with the tubes 150 may be referred to as the “fingertips”. Optionally, the fingertips may be removed from the remainder of the fingers 115, so that they may be cleaned without replacing the entirety of the fingers 115. In order to enable this, the fingertips may be plugged and unplugged from the remainder of the fingers 115 using any suitable releasable connector. Additionally, the length of the fingers 115 may be increased to reduce ingress of contaminants from the fingertips into the remainder of the respective gripping mechanism 110. The range of motion of the fingers 115 may also be increased in order to create a larger opening for accommodating misalignment of the tubes 150 when gripped by the fingers 115.

FIGS. 3c and 3d depict a pair of jaws 112, 114 formed by an opposing pair of fingers 115 in the closed and open configurations, respectively. Each of the fingers 115 has a proximal end 115-1, a distal end 115-2 where the fingertips are located, and both fingers 115 rotate about a common pivot 115-3 located between the proximal end 115-1 and the distal end 115-2. An actuation mechanism moves the fingers 115 between the closed and open configurations. In this embodiment, the actuation mechanism includes a cam 116 located between the proximal ends 115-1 of the opposing pair of fingers 115. The fingers 115 may be resiliently biased towards the cam 116 using a resilient member 117 such as a spring 117, so that the cam 116 remains in contact with the proximal ends 115-1 of the fingers 115.

In the position shown in FIG. 3c, the cam 116 pushes the proximal ends 115-1 of the fingers 115 away from each other against the force of the resilient member 117. In this way, the distal ends 115-2 of the fingers 115 are pushed towards each other into the closed configuration via rotation about the pivot 115-3. When the cam 116 is in the position shown in FIG. 3d, the proximal ends 115-1 of the fingers 115 are moved towards each other, thereby moving the distal ends 115-2 of the fingers away from each other into the open configuration, via rotation about the pivot 115-3. The cam 116 may be rotated to any intermediate position so that the fingers 115 may be moved to any configuration between the open and the closed configurations. In this way, an opposing pair of fingers 115 may fully clamp the tube 150 in the closed configuration, or may fully release the tube 150 in the open configuration, or may lightly grip or engage the tube 150 at an intermediate configuration. The cam 116 may be driven rotationally using a motor 118, for example.

Either or both of the fingers 115 may have a seat portion 115-4 arranged to abut against the tube 150 when it is located between the fingers 115. In this way, a “reference” or “datum” is provided so that the tube 150 is consistently gripped in the same position between the fingers 115 when they are moved from the open configuration to the closed configuration. To ensure that the seat portion 115-4 contacts the tube 150 in both the open configuration and the closed configuration, the fingers 115 are arranged to overlap each other, as indicated by the dotted lines in FIGS. 3c and 3d. Additionally, both of the opposing fingers 115 may move simultaneously during rotation of the cam 116, which means that the tube 150 may remain centred throughout movement between the open and the closed configuration. This ensures that the tube 150 is consistently aligned between the fingers 115.

Each of the opposing pairs of fingers 115 may operate using an actuation mechanism as described above. Since each opposing pair of fingers 115 is actuated using a separate cam 116, each of the opposing pairs of fingers 115 may operate (e.g. open and close) independently of the other pairs of fingers 115 in a pre-determined manner. In other words, as each pair of fingers 115 is driven by a separate cam 116, the pair of fingers may be controlled (via the arrangement of the respective cam 116) to open and close at a different time (e.g. in a pre-determined sequence) to the other (e.g. adjacent) pairs of fingers 115. The sequence in which each pair of fingers 115 opens and closes may be pre-configured and fixed due to the separate cams 116 that control each opposing pair of fingers 115 being controlled by a common motor 118. With a small number of (e.g. three) pairs of fingers 115, the (e.g. three) corresponding cams 116 may between them provide a sufficient number of rest points to allow “on the fly” individual control of each pair of fingers 115. This may be achieved by rotating the common motor 118 that controls them all to a rest point on a cam 116 corresponding to a corresponding pair of fingers 115 being in the open configuration, while the other pairs of fingers 115 are in the closed configuration. Such individual control becomes more challenging as the number of pairs of fingers 115 (and hence corresponding cams 116) increases due to the increased number of possible open/closed configurations, and consequently rest points, required.

The motion of each pair of fingers 115 between the open and the closed configuration may be actuated in other ways, such as by using a linear screw or a linear actuator, or by moving the fingers 115 directly with individual servos, for example. In an alternative embodiment (not shown), the fingers 115 may be replaced by linear actuators.

The clamping of the tubes 150a, 150b by the gripping mechanisms 110a, 110b will now be described with reference to FIG. 3e. In operation, each of the fingers 115 of the first jaw 112 and the second jaw 114 are moved towards each other using the actuation mechanism described above, thereby squeezing and clamping the respective tube 150. Optionally, the actuation mechanism may first move the first jaw 112 and the second jaw 114 to an intermediate configuration to engage, but not clamp the tubes 150, so that the tubes 150 may be manipulated in the bioprocessing system 1. The part of each of the fingertips that contacts the tubes 150 preferably has a flat surface so that the tubes 150 are pinched uniformly by the gripping mechanisms 110a, 110b.

The opposing pairs of fingers 115 are preferably not moved simultaneously when gripping the tubes 150, i.e. they may be moved independently. The upstream pairs of fingers 115 may be moved first, beginning with those fingers 115 closest to the cutting plane 141 and ending with those fingers 115 furthest from the cutting plane 141. Subsequently, the downstream pairs of fingers 115′ may be moved. In embodiments where there are multiple pairs of downstream fingers 115′, these may also be moved starting with those closest to the cutting plane 141. In this way, any fluid contained in the tubes 150 can be removed from the gripped portion of the tubes 150 and instead pushed towards the consumable 13. This prevents any loss of fluid, and inhibits the fluid interfering with the welding process.

Optionally, the pumping unit may be operated so as to pump fluid upstream towards the consumable 13. This can help to ensure the tubes 150 are empty at the cutting plane 141, which helps the tubes 150 remain pinched shut.

In FIG. 3e, the tube 150a is depicted part-way through the above-described process, where each of the fingers 115a of the first gripping mechanism 110a are in different positions. In particular, the downstream fingers 115a′ are still open and the upstream fingers 115a are partway through moving. The tube 150b is depicted once the clamping process is completed, where all fingers 115b, 115b′ on the second gripping mechanism 110b are fully actuated towards each other. In this position, the tube 150b is fully pinched shut so that no fluid may pass through the gripped portion. The clamping of the tubes 150a, 150b may be performed simultaneously or may be performed at different times. For example, the robotic arm 3 may move the end effector 100 so as to clamp one of the tubes 150, before moving the end effector 100 so as to clamp the other of the tubes 150. If both tubes 150 are already present between the first jaw 112 and the second jaw 114 of both of the gripping mechanisms 110a, 110b, then the clamping may occur simultaneously.

Optionally, the robotic device 2 includes a force sensor, a torque sensor, and/or a compliance sensor (not shown) to ensure that the tubes 150a, 150b are not preloaded prior to welding. One or more of these sensors may be located in the robotic arm 3, in the end effector 100 or within one or both of the gripping mechanisms 110a, 110b. For example, if the force/torque/compliance sensor determines that the force exceeds a predetermined value (such as 10 Newtons) or that the tubes 150 are twisted, the gripping mechanisms 110a, 110b may release and re-grip one or both of the tubes 150a, 150b until the force measured is at a level suitable for welding. A machine vision system, which may comprise a camera (not shown) and a processing unit (not shown) may also be used to confirm whether the tubes 150a, 150b are engaged.

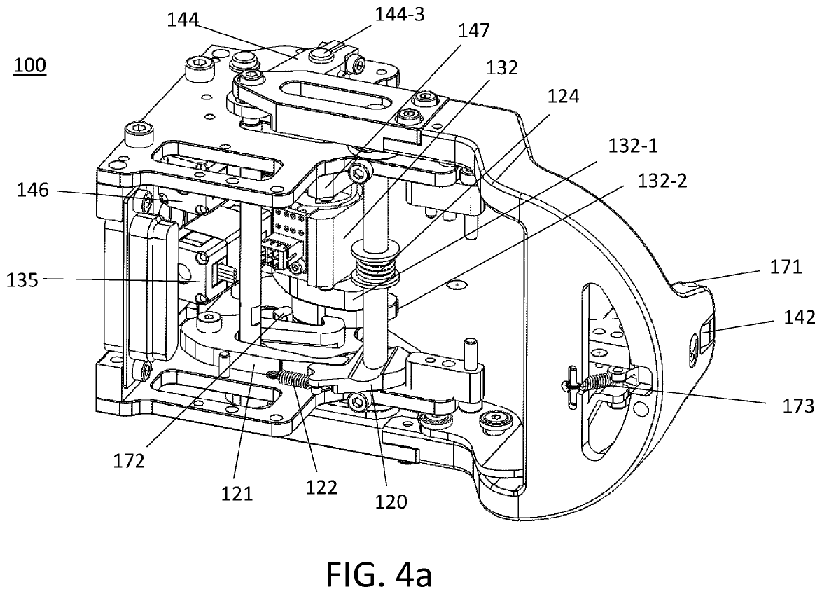

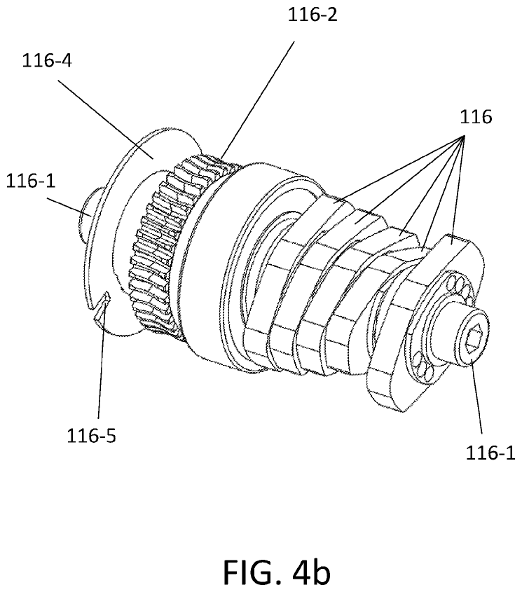

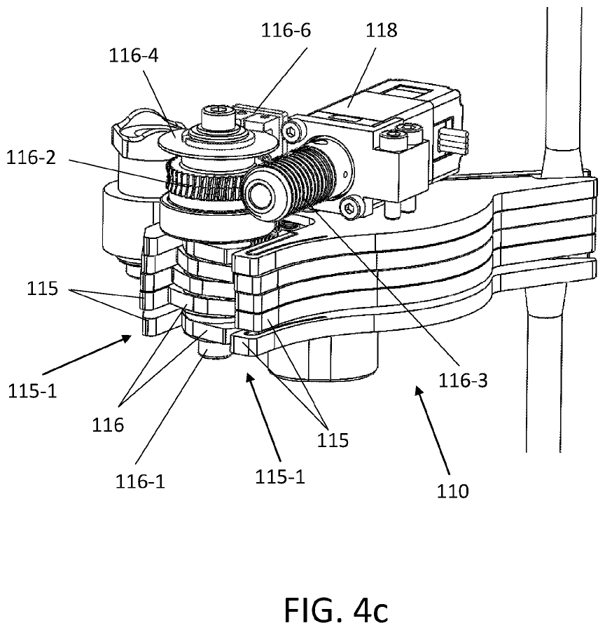

In FIG. 3f, the second gripping mechanism 110b may be moved towards the first gripping mechanism 110a so as to bring the tubes 150a, 150b closer together. In other words, the tubes 150a, 150b are moved in a direction substantially perpendicular to the longitudinal direction of the tubes 150a, 150b. In this example, rotation of a first cam 132-1 (visible in FIG. 4a) causes the second mounting plate 108 to rotate about a pivot (not shown), thereby moving the second gripping mechanism 110b towards the first gripping mechanism 110a. The second mounting plate 108 may be biased by a spring 134 towards the first cam 132-1 that causes it to rotate, in order to maintain contact therebetween. In this example, the first gripping mechanism 110a is static. Alternatively, the first gripping mechanism 110a may be moved towards the second gripping mechanism 110b, or both gripping mechanisms 110a, 110b may be moved towards each other. In other embodiments, neither of the gripping mechanisms 110a, 110b are moved.

Since both the first jaw 112b and the second jaw 114b of the second gripping mechanism 110b are mounted to the second mounting plate 108, they are moved at the same time without changing their relative position. In this way, the tubes 150a, 150b remain pinched shut and parallel to each other during the movement. A first stopper 130a (visible in FIG. 3i) is attached to the downstream finger 115a′ on the second jaw 114a of the first gripping mechanism 110a. A second stopper 130b is attached to the downstream finger 115b′ on the first jaw 112b of the second gripping mechanism 110b. When the first gripping mechanism 110a moves relative to the second gripping mechanism 110b, the first stopper 130a abuts against the second mounting plate 108. Similarly, the second stopper 130b abuts against the first mounting plate 106. In this way, the second gripping mechanism 110b is always moved to a consistent predetermined distance from the first gripping mechanism 110a.

Subsequently, the blade 140 may be heated to between 300° C. and 400° C. to sterilise and depyrogenate the blade 140. The blade 140 or a mounting block 142 in contact with the blade 140 may be heated using resistive heating, where electrical power is supplied to resistive elements in the blade 140 and/or the mounting block 142.

Alternatively, the blade 140 may be heated by a separate heat source or may be heated without direct contact such as through a laser heater. The blade 140 may be allowed to cool partially before it is used to cut the tubes 150. The power supply and/or the transformer for heating the blade 140 may be located outside (e.g. external to) the end effector 100 in order to reduce the weight and size of the end effector 100. For example, the power supply and/or the transformer may be located elsewhere in the robotic system 2, and wires may be used to transmit electrical power to the end effector 100. The power may also be used to operate other components such as the actuators or motors of the end effector 100. In this embodiment, the end effector 100 may weigh as little as 1.8 kg and may measure (e.g. have a “footprint” of) as little as 20 cm by 10 cm, thereby providing a substantial improvement on the size and weight of existing tube welders.

The movement of the blade 140 to cut the tubes 150a, 150b may be actuated in a number of ways. In this embodiment, FIG. 3f depicts the blade arm 145 in a retracted position where it does not affect operation of the gripping mechanisms 110a, 110b described previously. The blade arm 145 comprises a first endplate 143 that extends over an external face of the first baseplate 102. The blade arm 145 may also comprise a second endplate (not shown) that extends over an external face of the second baseplate 104. The actuation and motion of the second endplate may correspond to the actuation and motion of the first endplate 143, and therefore will not be described in detail.

On the first baseplate 102 is a pin 143-1 that extends into a slot 143-2 in the first endplate 143. In this way, the first endplate 143 may rotate about the pin 143-1 and may translate parallel to the first baseplate 102 in the direction of the slot 143-2. A linkage arm 144 is pivotally coupled to a proximal end of the first endplate 143 by a rod 144-1. The rod 144-1 may extend through a slot 144-4 in the linkage arm 144 and into a slot or groove 144-2 in the first baseplate 102. The slot or groove 144-2 may have a contour that constrains motion of the rod 144-1 along a particular path.

When driven by a motor 146 (visible in FIG. 4a) and preferably via a gearbox 147, the linkage arm 144 rotates about a shaft 144-3 to move the rod 144-1 through a curved portion of the slot 144-2. This motion causes the first endplate 143 to initially rotate about the pin 143-1 thereby swivelling the blade arm 145 so that the mounting block 142 and the blade 140 are located adjacent to the tubes 150a, 150b. During continued rotation of the linkage arm 144, the rod 144-1 moves through a linear portion of the slot 144-2. This moves the first endplate 143 in a linear direction where the pin 143-1 moves through the slot 143-2 in the first endplate 143. This translates the first endplate 143 and the blade arm 145 so that the blade 140 moves along the cutting plane 141 and intersects the tubes 150a, 150b. The final position of the blade arm 145 is shown in FIG. 3g, where arrows indicate the above-described motion of the blade arm 145.

As a result, the first tube 150a can be cut into an upstream section 150a connecting to the consumable 13 and a downstream section 150a′ which previously formed the end of the tube 150a. Similarly, the second tube 150b can be cut into an upstream section 150b connecting to the consumable 13 and a downstream section 150b′ which previously formed the end of the tube 150b. In this way, the new ends of the upstream parts 150a, 150b, formed by removal of the downstream sections 150b, 150b′, share a common plane.

The blade 140 may be a copper wafer with high thermal conductivity to allow the temperature of the blade 140 to be quickly changed. The blade 140 may be replaced between each tube welding operation to ensure a clean surface for every weld. In order to replace the blade 140, there may be a cartridge of wafers located in the mounting block 142 or elsewhere. Alternatively, the blade 140 may be provided by a ribbon of material so that a fresh portion of the ribbon may be moved into place after each welding operation.

In FIG. 3h, the upstream fingers 115a, 115b have moved so as to bring the upstream sections of the tubes 150a, 150b into longitudinal alignment. In order to do so, the first cam 132-1 (not shown) that causes the second mounting plate 108 to rotate continues to rotate, thereby pushing the second mounting plate 108 and the second gripping mechanism 110b towards the first gripping mechanism 110a. Due to abutment of the stopper 130b against the first mounting plate 106, further motion of the downstream fingers 115b′ of the second gripping mechanism 110b is prevented, but the upstream fingers 115b may continue to move.

As a result, the upstream section of the second tube 150b is moved out of alignment with the downstream section of the second tube 150b′ and into alignment with the upstream section of the first tube 150a. As this occurs, due to abutment of the stopper 130a (visible in FIG. 3i) against the second mounting plate 108, the downstream fingers 115a′ of the first gripping mechanism 110a are also pushed so that the downstream section of the first tube 150a′ is pushed out of alignment with the upstream section of the first tube 150a.

The blade 140 remains between the tubes 150a, 150b, and thereby transfers the heat from the heat source to melt the new ends of the tubes 150a, 150b. The blade 140 may be held between the tubes 150a, 150b for a predetermined time period and may have a predetermined heat profile. The predetermined time period and predetermined heat profile may be selected so that the tubes 150a, 150b are sufficiently melted for subsequent welding, but not hot enough to destroy the biological material contained in the tubes 150a, 150b or to compromise the structural integrity of the tubes 150a, 150b.

An infra-red camera or infra-red laser may be used in a closed loop to confirm that the ends of the tubes 150a, 150b have reached the correct temperature for welding and that a uniform temperature is reached. Alternatively, a thermistor, a thermocouple, or a resistance temperature detector (RTD) may be mounted on a component such as the blade 140, the mounting block 142 or the heat source in order to monitor the temperature. Alternatively or additionally, the resistance of the resistive elements may be directly measured in order to detect and control the temperature of the blade 140.

As depicted in FIG. 3h, the downstream fingers 115a′ (not shown), 115b′ gripping the downstream parts 150a′, 150b′ of the tubes 150a, 150b may remain in place. Optionally, the downstream fingers 115a′, 115b′ may be moved away from the upstream fingers 115a, 115b to prevent the upstream tubes 150a, 150b becoming accidentally welded to the downstream tubes 150a′, 150b′. If the upstream tubes 150a, 150b were to become accidentally welded to the downstream tubes 150a′, 150b′ then the force required to separate of the tubes 150 may tear the weld between the upstream tubes 150a, 150b. In order to prevent this, the tubes 150 are preferably held at least 4 mm apart.

Subsequently, the blade 140 can be removed from between the upstream fingers 115a, 115b and the downstream fingers 115a′, 115b′. As seen in FIG. 3i, the lifting mechanism 120 may be actuated to move the upstream fingers 115b of the second gripping mechanism 110b towards the upstream fingers 115a of the first gripping mechanism 110a. This position may be referred to a “raised” position. The downstream fingers 115a′, 115b′ may also be moved by the lifting mechanism 120. This movement presses the upstream part 150b of the second tube 150b into the upstream part 150a of the first tube 150a. Preferably, the lifting mechanism 120 is actuated sufficiently soon after removal of the blade 140 so that the ends of the tubes 150a, 150b are still molten when they contact. The lifting mechanism 120 may be any type of actuator that presses the upstream fingers 115b of the second gripping mechanism 110b towards the upstream fingers 115a of the first gripping mechanism 110a. For example, a linear actuator or a screw mechanism may be used to move either or both sets of upstream fingers 115a, 115b towards each other. The fingers 115a, 115b may be configured to move sequentially, or in a series of predetermined movements, for example.

In this embodiment the lifting mechanism 120 is a lifting pad 120 mounted to the second baseplate 104 of the end effector 100. As will be described later in relation to FIG. 4a, when actuated by a motor 135, the lifting pad 120 may be rotated relative to the second baseplate 104 and the upstream fingers 115a of the first gripping mechanism 110a. The rotation of the lifting pad 120 can be converted to linear motion of the second mounting plate 108 through a camming mechanism.

Since the upstream fingers 115b are mounted to the second mounting plate 108, linear motion of the second mounting plate 108 moves the upstream fingers 115b of the second gripping mechanism 110b towards the upstream fingers 115a of the first gripping mechanism. A spring 122 may be used to return the lifting pad 120 to its initial position. Additionally a spring 124 (visible in FIG. 4a) may bias the upstream fingers 115a, 115b away from each other once the lifting pad returns to its initial position. For example, the spring 124 may bias the second mounting plate 108 towards the second baseplate 104.

In this way, the position of the upstream fingers 115a of the first gripping mechanism 110a relative to the upstream fingers 115b of the second gripping mechanism 110b may be monitored in order to determine whether the weld is successful. For example, if a sensor (not shown) determines that the second mounting plate 108 returns to its initial position, then the weld is not strong enough to overcome the force from the spring 124, and thus is unsuccessful. On the other hand, if the weld is strong enough to retain the second mounting plate 108 in its “raised” position against the force of the spring 124, then the weld is successful.

The lifting mechanism 120 may equivalently be mounted to the first baseplate 102 and may instead move the upstream fingers 115a of the first gripping mechanism 110a via linear motion of the first mounting plate 106. In other embodiments, there may be a separate lifting mechanism 120 mounted both to the first baseplate 102 and the second baseplate 104. By actuating relative motion of the mounting plates 106, 108, there is no out-of-plane movement of the fingers 115. Alternatively, a lifting mechanism 120 may directly move the upstream fingers 115a, 115b relative to each other.

Once the lifting mechanism 120 is actuated to press the upstream tubes 150a, 150b into each other, the heat that was previously transferred to the tubes 150a, 150b by the blade 140 welds the upstream tubes 150a, 150b together so that they form a single tube 150. The joint or “interface” between the tubes 150a, 150b that have been joined together may further be referred to as a “butt weld”.

In FIG. 3j, the upstream fingers 115a, 115b may be partially retracted so that the tube 150 is still engaged between the fingers 115a, 115b but is not tightly clamped by the fingers 115a, 115b. Similarly, the downstream fingers 115a′, 115b′ may also partially retract to engage but not tightly clamp the downstream tubes 150b′, 150b′. The opposing pairs of fingers 115a, 115b that are closest to the cutting plane 141 may be moved fully into the open configuration to expose the butt weld between the tubes 150a, 150b. The opposing pairs of fingers 115a, 115b that are furthest from the cutting plane 141 may remain in position to engage the tube 150 and preferably to fluidly seal the butt weld from the sections of the tube 150 leading to the consumables 13. This means that even if the weld is unsuccessful, any fluid contained within the consumables 13 or the tubes 150 will not be exposed to the atmosphere.

Even when the butt weld is exposed by moving the upstream fingers 115a, 115b closest to the cutting plane, a portion of the tube 150 will still remain pinched at the connection between the upstream tubes 150a, 150b. This pinched portion may be referred to as a “kink” and must be removed before any fluid is able to pass through the tube 150. Before this occurs, the machine vision system may be used to inspect the connection between the two tubes 150a, 150b. By moving the upstream fingers 115a, 115b that are closest to the cutting plane 141 into the open configuration, part of the pinched portion of the tube 150 is exposed to allow for inspection.

The camera of the machine vision system preferably has a microscope lens and is connected to the processing unit (not shown), which identifies if a weld is successful. The camera may be located in any suitable location for observing the weld; depending on the location of the camera, one or mirrors may be included so that the camera may observe the weld via a reflection in the mirror. For example, a mirror may be located on the blade arm 145. The camera may be able to detect visible light, infra-red (IR) and/or ultra-violet (UV) radiation. The processing unit may examine the physical alignment of the tubes 150 to determine whether the weld is successful. Alternatively or additionally, it may inspect the “flash” around the weld to determine the integrity of the weld. The lifting pad 120 may be used to apply a tensile force to the tube 150 via the fingers 115, and may also provide measurement of the stress-strain profile of the tube 150. The stress-strain profile may also be analysed by the processing unit to confirm whether the weld is successful.

Other mechanical tests may be used, such as a torsion test or a vibration test, for example. An ultrasound source or X-ray source may also be used to test for the presence of cavities in the connection. Fluid may also be pumped through the tube 150, and the camera may be used to detect the presence of a leak.

Alternatively or additionally, a gas sensing method may be used to inspect the weld, where the connection is be located in a sealed container or volume. For example, the volume may initially be filled with an inert gas, and the container may have water vapour or oxygen sensors to detect any leakage through the weld into the volume. Alternatively, the volume may be filled with a tracer gas such as helium, which is subsequently removed from the volume. Then a sensor in the volume may detect leakage of the tracer gas back out of the weld. The container may be fitted with a pressure sensor that indicates a leak by detecting a pressure change inside the container. For example, if the container is initially pressurized, a decrease in pressure may indicate that gas is leaking into the weld. The camera may also be used to observe whether air leaks into the connection. Alternatively, air may be pumped into the tubes 150a, 150b prior to welding, and then a vacuum could be applied in the sealed container to see whether air leaks out.

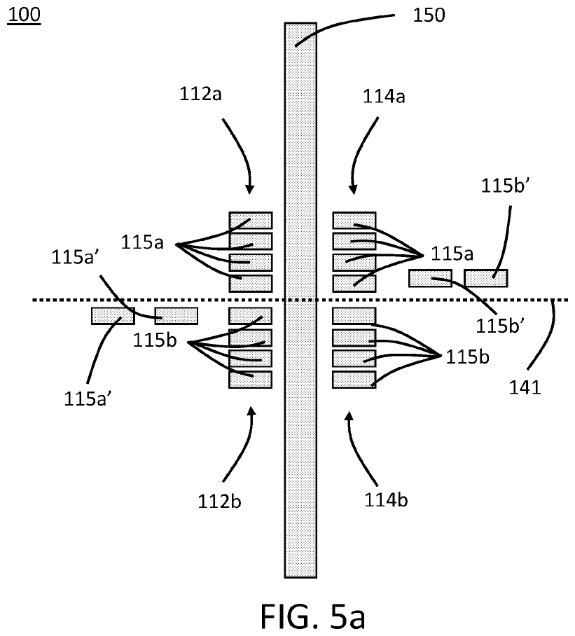

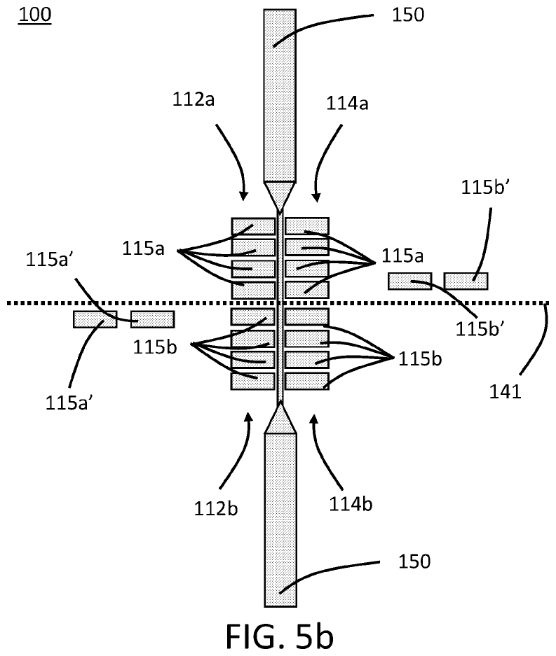

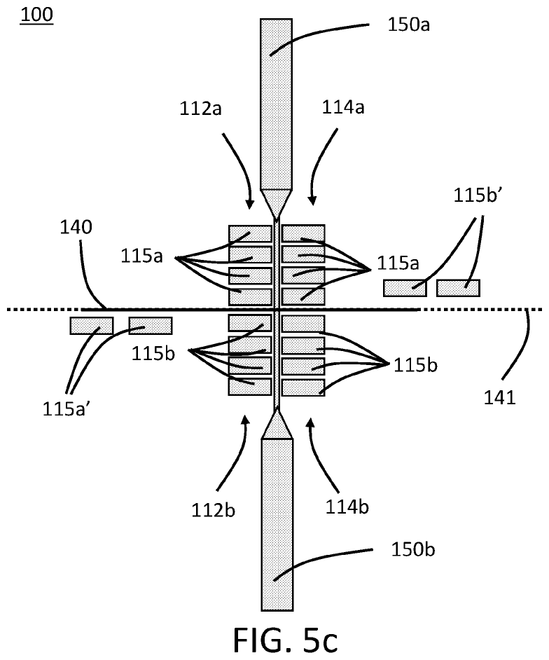

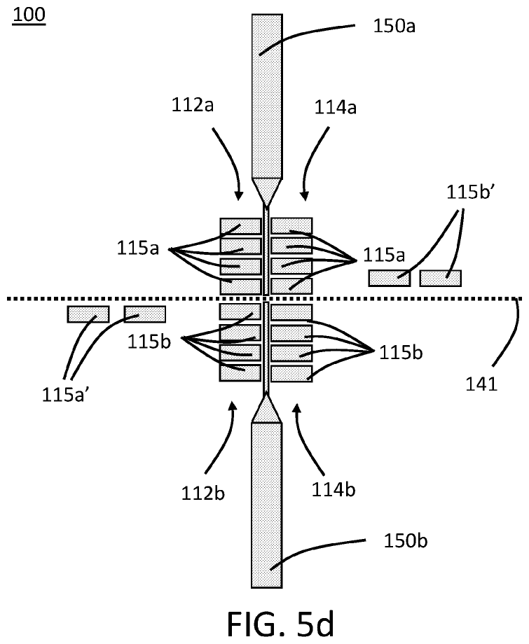

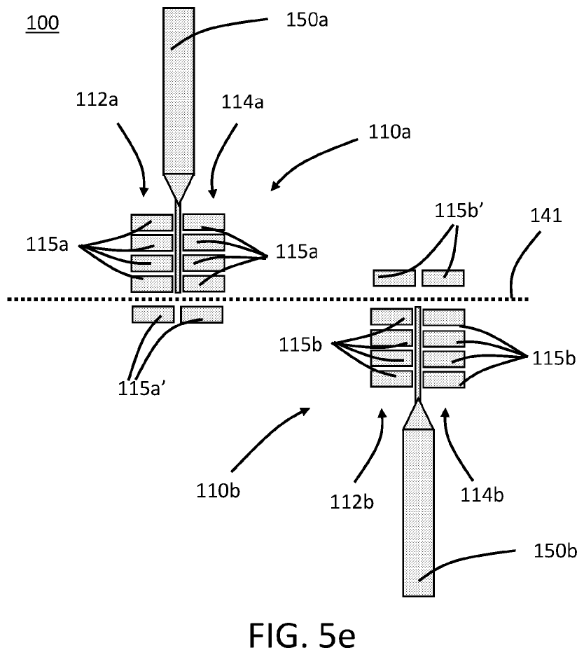

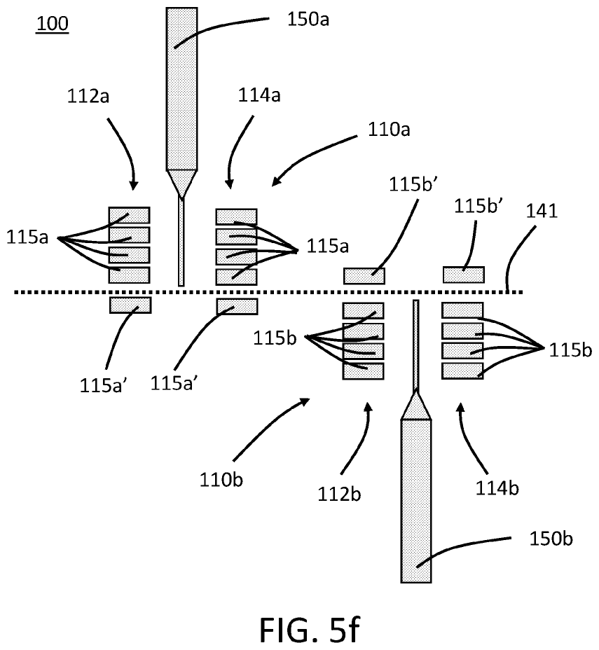

The inspection of the connection may be performed before the kink is removed from the tube 150. By inspecting the tube 150 before the kink is removed, even if a leak is present at the connection, the contents of the consumables 13 still remain isolated from the surrounding air and atmosphere. If the processing unit determines that the weld is not successful, the tube 150 may be re-clamped and re-welded. This may be performed in a number of ways; for example, the robotic device 2 may take the tubes 150a, 150b to an external clamp, before re-gripping at a different position further upstream to re-weld. Alternatively, the tubes 150a, 150b may be moved longitudinally through the gripping mechanism (as described further in relation to FIGS. 5a to 5c), so that the joint is formed further upstream.

In order to remove the kink (i.e. release the pinched portion of the tube 150) and open the tube 150, a pinch-release mechanism 170 may be used. The pinch-release mechanism 170 pushes against an edge of the pinched portion at the joint between the tubes 150a, 150b. Preferably, the pinch-release mechanism 170 pushes against the butt weld in a direction perpendicular to the direction in which the tube 150 is clamped by the jaws 112, 114, thereby causing the pinched portion to “pop open” due to Poisson's ratio effects.

There are a number of ways in which the pinch release mechanism 170 may be implemented. For example, there may be a separate arm with a popper plate, which moves along the cutting plane 141 to compress the tube 150 against the seat portion 115-4 of the fingers 115. There may be a separate set of jaws (e.g. comprising a first release element and a second release element), which grip and compress the edges of the pinched portion of the tube 150 to remove the kink. This pair of jaws may grip the tube 150 even when the tube is released by the gripping mechanisms 110a, 110b. As a further alternative, the blade 140 may be allowed to cool before being pressed against the butt weld through actuation of the blade arm 145.

In this embodiment, the pinch-release mechanism 170 comprises a popper heel 170 located on the mounting block 142. The mounting block 142 is connected to the blade arm 145 by a pin 171 about which the mounting block 142 may rotate. To operate the pinch release mechanism 170, the blade arm 145 swivels so that the mounting block 142 is adjacent to the pinched portion of the tube 150. The swivelling of the blade arm 145 occurs in the same manner as already described in relation to FIGS. 3f and 3g. As will be described later in relation to FIG. 4a, the mounting block 142 also rotates about the pin 171 so that the popper heel 170 is located adjacent to the tube 150. Subsequently, the blade arm 145 translates using the same mechanism as already described in relation to FIGS. 3f and 3g, thereby compressing the tube 150 between the popper heel 170 and the seat portion 115-4 of the fingers 115. Since the upstream fingers 115a, 115b closest to the cutting plane 141 have been moved into the open configuration, the popper heel 170 is able to fit between the opposing pairs of fingers 115a, 115b to compress the joint between the tubes 150a, 150b. Alternatively, the popper heel 170 may be a plate which fits in the gap between the upstream fingers 115a of the first gripping mechanism 110a and the upstream fingers 115b of the second gripping mechanism 110b.

In FIG. 3l, the blade arm 145 has been returned to its retracted position. The tube 150 remains lightly engaged by the upstream fingers 115a, 115b that are furthest from the cutting plane 141 and the downstream tubes 150a′, 150b′ remain engaged by the downstream fingers 115a′, 115b′. The inspection step described previously may also be performed after the kink in the tube 150 is removed, which may provide a better functional test for the tube 150. Preferably, the inspection step is performed both before the tube 150 is partially released by the upstream fingers 115a, 115b, and after the kink is removed from the tube 150 to allow fluid flow.

In FIG. 3m, the fingers 115 are fully moved to the open configuration to drop the tubes 150, 150a′, 150b′. This may occur simultaneously or the tubes 150, 150a′, 150b′ may be released separately so that they can be moved to separate locations before being released. The downstream tubes 150a′, 150b′ remain closed at both ends and thus may be discarded without the inside of the downstream tubes 115a′ 115b′ being exposed to the surroundings. The downstream tubes 150a′, 150b′ may be discarded into a particular container in the bioprocessing system 1, such as a container for waste disposal. The tube 150 between the two consumables 13 may also be placed by the end effector 100 at a particular location in the bioprocessing system 1. This may occur to allow the robotic system 2 to perform other steps in the bioprocessing method, and to allow the tube 150 to be quickly located later. Now that the connection between the original tubes 150a, 150b is complete, the pumping unit can be operated to pump fluid through the tube 150 between the consumables 13 in order to perform a step in the cell therapy process.

Optionally, the tube 150 may remain engaged by the end effector 100 during pumping.

A rear view of the end effector 100 is shown in FIG. 4a, where components such as the mounting plates 106, 108 and the gripping mechanisms 110 have been removed for clarity. The motor 146 that drives rotation of the shaft 144-3 and the linkage arm 144 via the gearbox 147 is shown. Once actuated this motor 146 is used to swivel and translate the blade arm 145 in the manner already described. Also depicted is the first cam 132-1 which causes movement of the second gripping mechanism 110b towards the first gripping mechanism 110a, as described previously. The first cam 132-1 is rotated by the motor 135 via a gearbox 132. In this embodiment, the motor 135 is a different motor to the motor 146, and the gearbox 132 is different to the gearbox 147. A second cam 132-2 is also driven by the motor 135 via the gearbox 132. The second cam 132-2 is used to actuate both the lifting mechanism 120 and to rotate the mounting block 142 so that the popper heel 170 may be located adjacent to the tube 150. Due to the profile of the second cam 132-2, both the lifting mechanism 120 and the mounting block 142 may be actuated only using rotation of the second cam 132-2. Alternatively, separate cams and/or motors may be used to actuate each of the lifting mechanism 120 and the mounting block 142.

For actuation of the lifting mechanism 120, the second cam 132-2 pushes against a lift plate drive arm 121. The lift plate drive arm 121 pushes against the lifting pad 121 against the force of the spring 122 as already described previously in relation to FIG. 3i. For rotation of the mounting block 142, the second cam 132-2 pushes against a popper-engaging arm 172, which rotates the mounting block 142 against the force of a spring 173.

FIG. 4b shows a series of cams 116 arranged along a camshaft 116-1 having a worm gear wheel 116-2 mounted thereon for receiving rotational drive input from the motor 118. Also located on the camshaft 116-1 is an alignment disc 116-4 with a notch 116-5. The location of the notch 116-5 may be detected by an alignment detector 116-6 in order to determine the rotational position of the camshaft 116-1 and the cams 116. Additional notches, a series of notches and/or additional alignment detectors 116-6 may also be present so that the positions of the fingers 115 may be confirmed. For example, the alignment detector 116-6 may confirm whether the fingers 115 are in the fully gripped position, the fully open position, or any combination. FIG. 4c shows the camshaft 116-1 and cams 116 arranged to drive a gripping mechanism 110, whereby the worm gear wheel 116-2 is arranged to receive rotational drive input from the motor 118 arranged to output rotation via a worm gear 116-3. During rotation of the cams 116 by the motor 118, the fingers 115 move between the open and closed configurations in the manner described previously. Both of the gripping mechanisms 110a, 110b are actuated as described above using separate motors 118. Alternatively, the gripping mechanisms 110a, 110b may be actuated by a common motor, for example.

The above method may be improved as follows to further minimise the chance of any leaks when the tubes 150 are cut. Once the tubes 150a, 150b are first gripped by the gripping mechanisms 110a, 110b, the lifting mechanism 120 may be actuated to increase the distance between the upstream fingers 115a, 115b and the downstream fingers 115a′, 115b′, thereby applying tension to the tubes 150a, 150b. Then once the tubes 150a, 150b are cut by the blade 141 and the upstream tube 150a, 150b are aligned, the lifting mechanism 120 decreases the distance between the upstream fingers 115a, 115b to press the tubes 150a, 150b into each other (i.e. provide compression between the tubes 150a, 150b) to weld them together. Preferably, the lifting mechanism 120 may move the fingers 115 through a range of positions and may apply a predetermined force. For example, the lifting mechanism 120 may be configured to have at least three predetermined positions: an initial baseline position in which a tube 150 is simply held, a position in which a clamped tube 150 is stretched, and a position in which the ends of two tubes 150a, 150b are pressed together.

The disconnection process of two consumables 13 (e.g. reconfiguration of the tubes 150) will now be described with reference to FIGS. 5a to 5f, which depict the fingers 115 of the end effector 100 shown in FIG. 3b. In FIG. 5a, a welded tube 150 connecting two consumables 13 is located between the upstream fingers 115a, 115b of the first and second gripping mechanisms 110a, 110b, but the gripping mechanisms 110a, 110b are not yet actuated to pinch the tube 150. The downstream fingers 115a′, 115b′ are not in use during the disconnection process.

In FIG. 5b, the upstream fingers 115a, 115b are moved to grip and pinch shut the tube 150. Similarly, to the connection process, the upstream fingers 115a, 115b may engage (and thereby pinch) the tube 150 at different times. For example, the upstream fingers 115a, 115b that are closest to the cutting plane 141 may be moved first followed by those furthest from the cutting plane 141. In this way, any fluid contained in the tube 150 can be removed from the gripped portion of the tube 150 and instead be pushed towards one of the consumables 13. This prevents any loss of fluid and inhibits the fluid interfering with the welding process.

Subsequently, the blade 140 is heated by the heat source (not shown) to between 300° C. and 400° C. to sterilise and/or depyrogenate the blade 140. The heating profile used during disconnection may be different to the heating profile used during connection, in order to better seal the tube 150. The blade 140 is allowed to cool partially. Similarly to the connection process, the blade 140 is moved along the cutting plane 141 to intersect the tube 150, as shown in FIG. 5c. This cuts the tube 150 into a first tube 150a connecting to a first consumable 13 and a second tube 150b connecting to a second consumable 13. The blade 140 remains between the tubes 150a, 150b for a predetermined time period to melt the ends of the tubes 150a, 150b, and thereby seal them. The predetermined time period used to melt the ends of the tubes 150a, 150b during disconnection may be different to the predetermined time period used to melt the ends of the tubes 150a, 150b during connection. The predetermined time period may be longer during the disconnection process. In FIG. 5d, the blade 140 is removed from between the first tube 150a and the second tube 150b.

Alternatively, it may be desirable to separate the sealing and cutting functions and seal the tube 150 to be disconnected over a wider length than can be achieved just using the blade 140. The tube 150 will first be sealed either by application of heat or RF energy generated at the fingertips. In order to heat seal the tube 150, individual fingertips may be made from resistive elements and supplied with power while compressing the tube 150, with the heat conducting through the tube 150 and melting the inside surfaces together. Preferably the fingertips will be coated with a non-stick material. Alternatively, individual fingers 115 may be used as a means for providing RF heat sealing. The fingertips may be made from ceramic and supplied with an alternating current operating in a MHz or GHz frequency range, preferably at 40.68 MHz. The RF method is advantageous as the tube material melts across a wider area and with more uniform temperature distribution through the cross section of the tube 150, therefore allowing a wider and more robust seal of the tube 150.

In FIG. 5e, the upstream fingers 115a of the first gripping mechanism 110a are moved away from the upstream fingers 115b of the second gripping mechanism 110b, thereby separating the first tube 150a from the second tube 150b. In doing so, the upstream fingers 115a, 115b realign with the downstream fingers 115a′, 115b′.

In FIG. 5f, the tubes 150a, 150b are released by the first and second gripping mechanisms 110a, 110b. The releasing of the tubes may occur simultaneously, or may occur at different times. For example, the first and second tubes 150a, 150b may be moved to different locations by the robotic arm 3 before being released by the first and second gripping mechanisms 110a, 110b. This may be to facilitate subsequent steps in the cell therapy method. Optionally, the first and second tubes 150a, 150b may be released before the tubes are separated. The upstream fingers 115a, 115b may therefore be released after removal of the blade 140 but before the upstream fingers 115a, 115b are aligned with the downstream fingers 115a′, 115b′.

The connection and disconnection processes described above may be performed as many times as required for a particular bioprocessing method. During each connection step the portions of the downstream tubes 150a′, 150b′ are discarded, so the length of the tube 150 between the downstream end and the consumable 13 will get shorter over time. To account for this, a spare supply of tube 150 may be provided in the bioprocessing system 1, and the end effector 100 may be operated to extend the existing tubes 150 by welding them to tubes from the spare supply. Optionally, the spare supply of tube may be integrated directly into the end effector 100 so that fresh tubing is always available when a tube 150 needs to be extended. Preferably, during the connection process the gripping mechanisms 110a, 110b grip the tubes 150a, 150b close to the downstream end, so that the length of discarded downstream tube 150a′, 150b′ is minimized.

The gripping mechanisms 110a, 110b may also be operated to move the tubes 150 longitudinally while they are held by the gripping mechanisms 110a, 110b. The term “longitudinally” refers to a direction parallel or “coaxial” to the axis of the tubes, which enables the location on the tube 150 gripped by the gripping mechanisms 110a, 110b to be adjusted to be closer to the respective consumable 13 or closer to the downstream end of the tube 150. This increases the degree of control of the gripping mechanisms 110a, 110b, and reduces the size of the discarded downstream tubes 150a′, 150b′ that are produced during the connection process. The longitudinal movement of the tube 150 is achieved in a manner similar to an inch worm drive, where the tube 150 is gradually shuffled through the gripping mechanism 110 without the gripping mechanism ever fully releasing the tube 150.

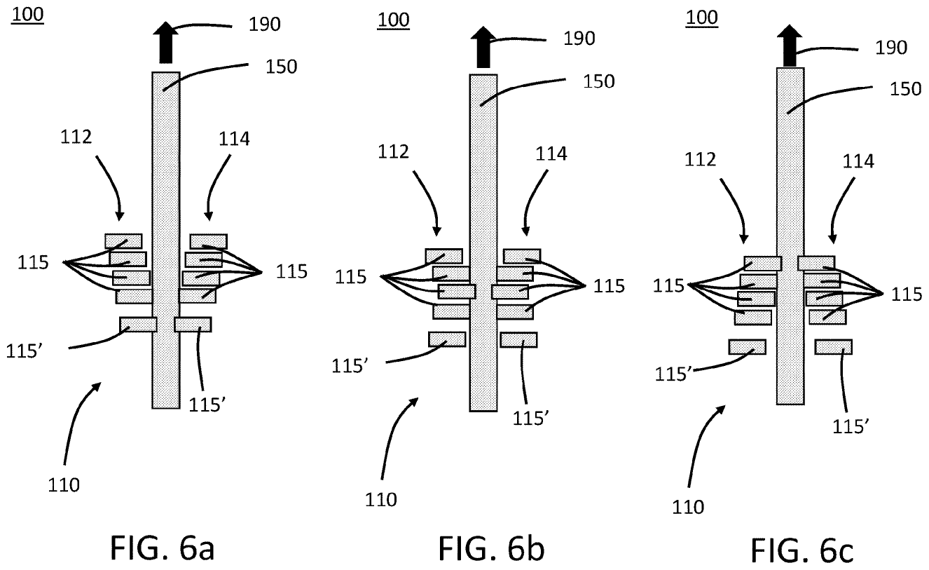

With reference to FIGS. 6a to 6c, the longitudinal movement of a tube 150 will now be described. Only one of the gripping mechanisms 110 is shown, but it will be appreciated that both of the gripping mechanisms 110 may enable longitudinal movement, and may do so simultaneously.

In order to move the tubes 150 longitudinally, the fingers 115 periodically grip and release the tube 150 out of phase with each other, for example sequentially or in a predetermined sequence. FIG. 6a depicts the gripping mechanism 110 at a first stage in the process, the downstream fingers 115′ are moved together to squeeze the tube 150. The downstream fingers 115′ may fully pinch the tube 150 or may be in a position where the tube 150 is lightly squeezed but not fully pinched.

The upstream fingers 115 become progressively further apart in the direction away from the downstream fingers 115′. FIG. 6b depicts the gripping mechanism 110 at a later stage in the process, where the downstream fingers 115′ have begun to move apart, and now one opposing pair of the upstream fingers 115 is squeezing the tube 150. The opposing pair of upstream fingers 115 may fully pinch the tube 150 or may be in a position where the tube 150 is lightly squeezed but not fully pinched. FIG. 6c depicts the gripping mechanism 110 at a still later stage in the process, where the downstream fingers 115′ have moved apart, and the upstream fingers 115 at the opposite end of the gripping mechanism 110 are now squeezing the tube 150.

When the steps shown in FIGS. 6a to 6c are repeated many times, the tube 150 will move in the direction of the arrow 190. This motion may be referred to as a driven wave of fingers 115 pinching the tube 150. By reversing the process described above, the tube 150 will move in the opposite direction. The motion of each of the fingers 115, 115′ may be a sinusoidal motion, and adjacent fingers 115, 115′ may be offset by a particular phase angle. This phase angle may depend on the total number of fingers 115, 115′ present in the gripping mechanism 110.

Alternatively, longitudinal motion of the tube 150 may be achieved by using the lifting mechanism 120. At a first stage, the upstream fingers 115 may initial grip (but not fully pinch) the tube 150. At a subsequent stage, the downstream fingers 115′ grip the tube 150 and the upstream fingers 115 are retracted. Then the lifting mechanism 120 is actuated to move the (closed) downstream fingers 115′ closer to the (opened) upstream fingers 115. Then the upstream fingers 115 grip the tube 150 again, and the downstream fingers 115′ release the tube 150. Subsequently, the lifting mechanism 120 is turned off so that the (opened) downstream fingers 115′ move away from the (closed) upstream fingers 115. Thus, the gripping mechanism 110 returns to its original position, but with the tube 150 moved longitudinally relative to it. This may be repeated many times to move the tube 150 through the gripping mechanism 110. The process may be reversed to move the tube 150 in the opposite direction.

When the gripping mechanism 110 is operated to move the tube 150 longitudinally, the pinch release mechanism 170 may be positioned in front of the fingers 115 to retain the tube 150 in the gripping mechanism 110. Alternatively, the gripping mechanism 110 may be moved to a particular orientation when the tube 150 is moved longitudinally so that the tube 150 does not fall out of the gripping mechanism 110.

Any features described in relation to the end effector 100 described herein may be implemented in a static tube welding device. For example, the pinch release mechanism 170 may be combined with any tube welder, whether located on a robotic arm 3 or otherwise, and may be supplied as a separate component. The tube welder may have a machine vision system or camera as described herein to confirm whether the tubes are engaged correctly. The machine vision system or camera may also be used before and/or after unpinching the tube 150 to confirm whether the weld is successful. Such a tube welder may require manual operation (or a separate robotic device) to locate the tubes between the jaws 112, 114. However, subsequent steps such as clamping, cutting, alignment, inspection and operating the pinch-release mechanism 170 may be operated autonomously by the tube welder.

Similarly, the gripping mechanisms 110a, 110b with a plurality of pairs of gripping elements 115 may be supplied or used separately, in both manual and automated tube welders, whether static or located on an end effector 100. Such gripping mechanisms 110a, 110b may be operated in accordance with any of the steps described herein, such as for longitudinal movement of the tube 150, or for removing fluid from near the cutting plane 141.

As used herein, the gripping mechanisms 110a, 110b may collectively be referred to as a “clamping unit”. The first jaw 112a, 112b of each of the gripping mechanisms 110a, 110b may equivalently be referred to as the first jaw of the clamping unit. Similarly, the second jaw 114a, 114b of each of the gripping mechanisms 110a, 110b may equivalently be referred to as the second jaw of the clamping unit.

The upstream fingers 115a of the first jaw 112a of the first gripping mechanism 110a and the downstream finger 115b′ of the first jaw 112b of the second gripping mechanism 110b may collectively be referred to as the first part of the first jaw. The downstream finger 115a′ of the first jaw 112a of the first gripping mechanism 110a and the upstream fingers 115b of the first jaw 112b of the second gripping mechanism 110b may collectively be referred to as the second part of the first jaw. The upstream fingers 115a of the second jaw 114a of the first gripping mechanism 110a and the downstream finger 115b′ of the second jaw 114b of the second gripping mechanism 110b may collectively be referred to as the first part of the second jaw. The downstream finger 115b′ of the second jaw 114a of the first gripping mechanism 110a and the upstream fingers 115b of the second jaw 114b of the second gripping mechanism 110b may collectively be referred to as the second part of the second jaw.

While the foregoing is directed to exemplary embodiments of the present invention, it will be understood that the present invention is described herein purely by way of example, and modifications of detail can be made within the scope of the invention. Furthermore, one skilled in the art will understand that present invention may not be limited to the embodiments disclosed herein, or to any details shown in the accompanying figures that are not described in detail herein or defined in the claims. Indeed, such superfluous features may be removed from the figures without prejudice to the present invention. It will also be appreciated that particular combinations of the various features described and defined in any aspects described herein can be implemented and/or supplied and/or used independently. Any apparatus feature described herein may also be incorporated as a method feature, and vice versa.

Moreover, other and further embodiments of the invention will be apparent to those skilled in the art from consideration of the specification, and may be devised without departing from the basic scope thereof, which is determined by the claims that follow.

1-30. (canceled)

31. An automated apparatus for joining a flexible tube to another such tube whereby to form a fluidic path therethrough, comprising:
means for engaging the flexible tube and moving it into one or more positions to be manipulated;
means for clamping a portion of the flexible tube whereby to form a pinched portion of the tube such that the flexible tube is fluidly sealed upstream of the pinched portion;
means for removing an end section of the flexible tube downstream of the pinched portion such that a new end of the flexible tube is thereby formed within the pinched portion that has not previously contacted another such tube; and
means for joining the pinched portion at the new end of the flexible tube with a corresponding pinched portion of another such tube;
wherein the apparatus is configured as an end effector for a robotic device.

32. The apparatus of claim 31, wherein the end effector is configured to receive electrical power from an external power supply via an electrical coupling provided therebetween.

33. The apparatus of claim 31, further comprising a sensor configured to determine whether at least one of the first tube and the second tube are engaged or aligned in the means for engaging the tube.

34. The apparatus of claim 31, further comprising means for manipulating the tube, once joined with said another such tube, to release the pinched portion whereby to establish a fluidic path between the joined tubes.

35. The apparatus of claim 34, wherein the means for manipulating the tube comprises an automated pinch-release mechanism arranged to press on an edge of the pinched portion of the tube thereby to release the pinched portion and open up the welded tubes whereby to form a fluidic path therethrough.

36. The apparatus of claim 35, wherein the pinch-release mechanism is arranged to compress the pinched portion of the tube against a seat portion of one or more of the means for engaging and moving the tube and the means for clamping a portion of the tube.

37. The apparatus of claim 35, wherein the pinch-release mechanism comprises a first release element and a second release element, the first and second release elements arranged to engage with opposing edges of the pinched portion of the tube at an interface between the new ends of the first and second tubes when joined together.

38. The apparatus of claim 35, wherein the pinch-release mechanism is arranged to press on an edge of the pinched portion by moving along a straight path.

39. The apparatus of claim 31, further comprising means for stretching the interface between the new ends of the first tube and second tube when joined together to verify the integrity of the join.

40. The apparatus of claim 31, further comprising means for visually inspecting the interface between the new ends of the first tube and second tube, when joined together, to verify the integrity of the join.

41. The apparatus of claim 31, wherein the means for engaging a tube is provided by a tube gripping mechanism comprising a pair of jaws operable for relative movement between an open configuration for receiving the tube therebetween and a closed configuration for engaging the tube therebetween.

42. The apparatus of claim 41, wherein the means for clamping a portion of the tube is also provided by the tube gripping mechanism, where the closed configuration of the pair of jaws is configured to clamp the tube therebetween.

43. The apparatus of claim 42, wherein the tube gripping mechanism comprises a plurality of pairs of gripping elements arranged adjacently, each pair of gripping elements comprising a first gripping element and a second gripping element, and each pair of gripping elements being configured for movement between said open and closed configurations.

44. The apparatus of claim 43, wherein a first pair of gripping elements and an adjacent second pair of gripping elements are spaced apart to provide a gap for receiving therebetween means for removing an end section of the tube.

45. The apparatus of claim 43, wherein a first pair of gripping elements is configured to engage a first portion of the end section of tube before a second pair of gripping elements is configured to engage an adjacent second portion of the end section of the tube, whereby to urge fluid in the tube away from the first portion of the tube towards the second portion of the tube prior to operation of means for removing an end section of the tube arranged to remove a portion of the end section of the tube on the other side of the first pair of gripping elements.

46. The apparatus of claim 43, wherein the gripping mechanism is controllable to clamp different parts of said portion of tube while releasing other parts of said portion of tube.

47. The apparatus of claim 46, wherein the gripping mechanism is controllable to retain the portion of tube between at least one pair of gripping elements in the closed configuration while at least one different pair of gripping elements are moved to the open configuration to release part of said portion of tube such that said part of said portion of tube is exposed.

48. The apparatus of claim 47, further comprising means for inspecting said released or exposed part of said portion of tube.

49. The apparatus of claim 47, wherein means for manipulating the tube to release the pinched portion is arranged to compress said released or exposed part of said portion of tube.

50. The apparatus of claim 43, wherein the plurality of pairs of gripping elements of the gripping mechanism are controllable to move an engaged tube in a direction substantially perpendicular to a longitudinal length of the tube relative to the gripping mechanism.
